(19) United States
(12) Patent Application Publication
(10) Pub. No.: US 2015/0117603 A1
(43) Pub. Date: Apr. 30, 2015

(54) DETECTOR ASSEMBLY FOR RECORDING X-RAY IMAGES OF AN OBJECT TO BE IMAGED

(71) Applicants: Fraunhofer-Gesellschaft zur Förderung der angewandten Forschung e.V., München (DE); Charite-Universitätsmedizin, Berlin (DE)

(72) Inventors: Erwin Keeve, Potsdam (DE); Sebastian Engel, Munster (DE); Fabian Stopp, Berlin (DE); Marc Käseberg, Biesenthal (DE); Eckart Uhlmann, Kiebitzreihe (DE)

(21) Appl. No.: 14/384,470

(22) PCT Filed: Mar. 15, 2013
(86) PCT No.: PCT/EP2013/055458

§ 371 Date: Sep. 11, 2014

(30) Foreign Application Priority Data
Mar. 15, 2012 (DE) 10 2012 005 899.3

(51) Int. Cl. A61B 6/00; A61B 6/04

(52) U.S. Cl. CPC A61B 6/4458; A61B 6/4233; A61B 6/4405; A61B 6/4452; A61B 6/4464; A61B 6/0407
USPC 378/62; 378/189

(57) ABSTRACT

The invention relates to a detector assembly for recording x-ray images of an object to be imaged, said object being located on a support plate of a table, wherein the table has at least one adjustable support foot for moving the support plate, comprising: a detector for detecting x-radiation and a positioning device for moving the detector relative to the object, wherein the detector can be moved into a plurality of recording positions that are spatially fixed with respect to the object and the positioning device can be fastened to the support foot in such a way that the positioning device is moved together with the support plate and the positioning device has at least one articulated arm, which can be moved with respect to at least one axis of rotation and/or one linear axis.

The present invention relates to a detector arrangement for recording X-ray pictures of an object to be imaged, as well as to an X-ray device which comprises such a detector arrangement, and to a method for recording X-ray pictures.

X-ray devices serve for the destruction-free examination of objects to be examined. X-ray devices in the field of medicine are particularly applied for examining patients. X-ray radiation which is emitted by an X-ray source of the X-ray apparatus hereby partially penetrates the object to be examined, wherein absorption of the X-ray radiation occurs to a different extent depending on the through-radiated material. A share of the X-ray radiation which passes through the object is detected by a detector as a projection picture of the transilluminated object. Spatial information is represented in a superimposed manner in the projection picture, whereas a three-dimensional representation for example of interior of the body permits an exact reposition of bone breakages or fractures on joints or an exact positioning of implants relative to critical anatomical structures.

Several two-dimensional projection recordings of an object from different spatial directions are taken with the X-ray device, and the scanned volume is subsequently reconstructed by way of an algorithm, for producing three-dimensional image data. Computer tomographs are applied for this purpose and although permitting a very good picture quality, they are however unsuitable for inter-operative application, since here the access to the patient is blocked by the apparatus. Additionally, such apparatus require a lot of space, have a high radiation exposure, are complex in operation and entail high costs. Generally, until now there has existed no possibility of travelling a path of more than 180° close to the patient without parts which are moved and are therefore dangerous.

It is the object of the present invention, to suggest a detector arrangement which avoids the mentioned disadvantages, with which therefore several X-ray recordings can be made from arbitrary positions, with an as small as possible detector, wherein an object to be imaged is accessible between the recordings.

According to the invention, this object is achieved by a detector arrangement according to patent claim 1. Advantageous further developments are described in the remaining claims.

Patent claim 1 relates to a detector arrangement for recording X-ray pictures of an object which is to be imaged and is located on a rest of a table, wherein the table comprises at least one adjustable support foot for moving the rest, comprising: a detector for detecting an X-ray radiation and a positioning device for moving the detector with respect to the object, wherein the detector is movable into several recording positions spatially fixed with respect to the object, and the positioning device is fastenable on the support foot in a manner such that the positioning device is moved together with the rest, and the positioning device comprises an articulated arm which is movable via at least one rotation axis and/or a linear axis.

Huge advantages result from the fact that the positioning device for moving the detector is regularly co-moved with the rest. A quality and simplicity of the picture recording of an object (preferably a human patient) lying on the rest is rendered possible due to this. Moreover, the spatial requirement of such an arrangement is minimal, since less standing surface is taken up by the positioning device and the detector in the operating room.

A further development hereby envisages the positioning device being fastenable between the support foot and rest. This has the advantage that the positioning device can be assembled as an additional component between an existing support foot and an existing rest. For this reason, it is not necessary here to view a “complete unit” (consisting of rest, positioning device, support foot and possibly X-ray source) as the smallest unit capable of being marketed, but instead such a positioning device can also be envisaged for retrofitting existing systems.

A further development moreover envisages the rest at least in regions consisting of a material which is transparent to X-rays, for example a carbon-fibre-reinforced plastic material (CFP material). Recordings can made directly below the rest in the idle position or initial position of the detector by way of this, in order to take X-ray recordings of a person located on the rest.

A further development envisages the support foot being designed such that the rest and positioning device together are adjustable in height and/or in inclination. Hereby, the height is of particular interest, in order to provide operating personnel of a different height with an optimal access to the patient or in order to accompany sitting or standing positions of operating person. It is also advantageous that an inclination of the rest can be provided, and this makes particular sense for the targeted influencing of the vessel pressure with certain medical indications. The inclination hereby can be adjusted in one or two spatial directions; for example a tilting of the patient to the operator (tilting about the longitudinal axis of the rest, indicated in the Figures as the “x-direction”, see FIG. 39ff) is possible. Moreover, a tilting about the y-axis (see FIG. 39ff.) is also possible (see above).

A further development envisages the rest being held on precisely one support foot. This is particularly usefully for reasons of space, since only a central column must be provided, and the standing surface around the rest/patient can thus be optimally utilised.

A further development envisages the rest and the positioning device being positioned with respect to the support foot in a manner such that an operator can accommodate his legs below the rest. It is also advantageous if the rest is linearly displaceable in its longitudinal direction with respect to the support foot, in order to render the spatial alignment and the space below the rest better utilisable. This movement can be effected together with the positioning device and/or independently of the positioning device. For example, it is possible for a co-guiding of the positioning device to be effected in unison with the rest; and additionally the detector itself may yet envisage a linear drive for “fine adjustment”.

A further development moreover envisages the positioning device comprising an articulated arm in the form of a robot arm with at least three robot arm axes, preferably however all four, five or six robot arms axes can be envisaged for positioning the detector. Hereby, it is advantageous if three of the robot arm axes are arranged parallel to one another. Very space-saving arrangements are possible by way of this, and moreover it is easily possible to run an arched, elliptical etc. path around the rest. In this manner, good spatial representations can be achieved without a very space-intensive C-arm having to be provided.

It is moreover advantageous if the detector, which is preferably attached at the end of the robot arm (synonymous with articulated arm), additionally to the rotation degrees of freedom by way of the robot arm axes, can yet additionally be positioned translatorily with respect to the rest.

It is to be emphasised that the movement of the detector or of the articulated arm in all spatial directions (i.e. about all rotation axes or in al linear directions) is effected motorically. However, it is also possible for non-motoric movements to be provided here and also to envisage the rotation joints or linear guides being self-locking, so that a position can be held without the provision of a motor. Amongst other things, it is also advantageous if the longitudinal direction of the rest is parallel to at least one robot arm axis, preferably parallel to two or three robot arm axes, since this is very favourable with regard to the spatial requirement of the articulated arm/robot arm.

It is also advantageous if the positioning device is designed in a manner such that the detector can be moved from at least two sides of the rest, from below the rest to above the rest. This ensures that an operator standing on one side of the rest is not inhibited by a positioning device/detector located on the same side

The detector arrangement described above can preferably be applied in an X-ray device which moreover yet comprises an X-ray source. Hereby, it can be useful for the X-ray source to be fastened on a further articulated arm/robot arm, wherein this articulated arm/robot arm is held on a ceiling of a room. This makes particular sense with regard to the space requirements as well as to the sterility requirements.

The interaction of the X-ray source with the detector arrangement is effected as described in the text below. Once again, it is to be emphasised that all features shown in the description, in particular all features shown in the general description introduction can be combined with one another inasmuch as this is not explicitly ruled out in the following text.

Further aspects or details of the patent application are as follows:

A detector arrangement for recording X-ray pictures of an object to be imaged comprises a detector, which serves for detecting X-ray radiation, and a positioning device for moving the detector with respect to the object to be imaged. The detector hereby is movable on an imaging surface along the positioning device into several recording positions which are spatially fixed with respect to the object. Moreover, the positioning device comprises at least one side element which can be angled with respect to the imaging surface.

The detector arrangement can be designed with a smaller construction size by way of the side element which can be angled, since the beaming-in of X-ray radiation from several sides at arbitrary angles is rendered possible, without the detector arrangement assuming an excessively large area. Different recording positions can be moved to, due to the movement of the detector on the imaging surface, and these positions despite this are spatially defined by the positioning device and permit an unambiguous assignment of X-ray images to positions of the detector. Thus projection pictures of the object to be imaged which in medicine is typically a patient, can be taken in a defined manner and be led to a further processing.

In a particularly preferred embodiment, the detector is arranged on an articulated arm as a positioning device, wherein the articulated arm is movable via at least one, preferably two, particularly preferably at least three articulation axes and/or a linear axis and/or has a combination of the at least one linear axis and/or at least one rotation axis. The articulated arm thus functions as an articulated mechanism. The detector itself can be arranged at an end of the articulated arm.

In an advantageous further development, the detector can be moved into the side element, so that recording positions situated in the side element can also be achieved without further mechanical modification of the detector arrangement.

The detector in at least one recording position situated on the side element can also lie completely outside the imaging surface. The detector arrangement turns out to be particularly space-saving during the recording on account of this, since the side element is bent or angled such that no part of the detector intersects the imaging surface, thus a width of the detector arrangement is minimised. “Imaging surface” hereby should be understood as a surface, on which or along which the detector can move, for example with its central point. The imaging surface hereby should not extend into the side element, but however can be continued via a side imaging surface in the side element. Typically, the detector arrangement however comprises two side elements which are located on parts of the detector arrangement which lie opposite one another. This permits a large region of incident angles of the X-ray radiation to be covered.

The at least one side element is preferably angled at an angle of 15° to 85°, preferably 30° to 70°, particularly preferably 40° to 60° with respect to the imaging surface in a middle part of the detector arrangement.

In a particularly preferred manner, the positioning device is U-shaped, so that the detector can be moved into several positions around a middle of the “U”. The element to be imaged is placed in the middle of the “U” and thus can be imaged from different directions.

The positioning device and the detector can be arranged within a housing. By way of this, these components are protected from mechanical damage and with operations for example, there is no danger that a physician or another person located in the room inadvertently damages one of the apparatus, or an undesired collision taking placed during an automated movement. Preferably, the housing comprises a material which is transparent to the X-ray radiation or consists completely of such a material. Likewise, the positioning device can also consist of a material which is transparent to X-ray radiation or comprise such a material. This permits a beaming-through of the housing and thus recordings from a greater angular region. The term “X-ray radiation” in the context of this document is to be understood as an electromagnetic radiation with a wavelength between 0.01 nm and 10 nm. In a particularly preferred manner, the housing comprises a closed surface, thus a surface without holes or openings, in order to minimise the danger to the patient or to keep the danger of a mechanical damage to the detector and to the positioning device to a minimum. Moreover, the positioning device and the detector are typically connected to one another.

In an advantageous further development, the detector arrangement is removably fastenable on a rest, with medical applications typically a table of an operating room, by way of a holder, wherein the object to be imaged lies on the rest. This fastening can be effected above as well as below or laterally of the rest. The detector arrangement can thus be attached onto different rests and be detached from the rests. Thus an access to the object to be imaged is given between the X-ray recordings, and the detector arrangement can be attached in a variable manner in different positions and attitudes.

Particularly preferably, the housing serves as a rest, so that the object to be imaged is held directly in front of the detector arrangement without further components.

The middle part of the detector arrangement which lies along the imaging surface, is typically fastened parallel to a plane which runs through the rest, inasmuch as the detector arrangement is fastened on the rest. This plane running through the rest is arranged horizontally in a particularly preferred manner. The middle part of the detector arrangement for this can be fastened above the rest, laterally to it or below it.

The side element preferably has a width which is smaller than a width of the middle part of the detector arrangement and/or is smaller than a width of the rest, in order to permit a space-saving construction manner. In further embodiments, the widths however can also be equally large, or the width of the side element can even be greater than the width of the middle part. The width of the rest can correspond to the width of the middle part.

The at least one side element can be removably attachable onto the positioning device, thus can be fastened on the positioning device as well as detached from this. Moreover, the detector arrangement can be folded away from the positioning device, for example by way of a hinge, or can be pulled out of the positioning device. The side element in particular can also be under the middle part and be pulled out for recording pictures. In the case of a side element which can be folded away, the side element is typically guided in its movement, for example by way of one or more rods. If the side element is not required, it can therefore be removed from the positioning device in a space-saving manner.

The side element preferably projects beyond a plane, which runs through the object to be imaged in a manner parallel to the rest. The detector can also be moved laterally to the object to be imaged by way of this, and pictures are obtained with a complete through-beaming of the object from the side. In a particularly preferred manner, the detector arrangement can also be folded to over the object to be imaged, thus encompass the object. Recordings of the object can be made at any angle by way of this.

In an advantageous further development, the positioning device comprises at least one rail system, on which the detector is mounted. This permits a mechanically stable mounting and movement of the detector. Preferably, several rail systems can be operated in a cascaded manner, thus several rail systems joined together, in order to permit a movement in a multitude of degrees of freedom. The rail systems can be linear, arcuate or freely shaped, at least in regions, in order to permit an as free as possible movement of the detector. Alternatively or additionally, the positioning device can also comprise a linear drive and/or the articulation mechanism, wherein a combination of the linear drive and the articulation mechanism is also possible.

One can envisage the positioning device being displaceable below, above and/or laterally to the object to be imaged. Elongate objects can also be imaged without any problem by way of this. For this, the positioning device can be arranged in a support foot of the rest or on a bearing guided below the rest. Alternatively, the positioning device can also be arranged on a displaceable vehicle.

The detector can be movable on the positioning device by way of the linear drive. In a further embodiment, the detector can be moveable by way of a rack and a pinion on a bearing guide of the positioning device. This permits a traversing or movement into precisely settable positions. Moreover, such arrangements are mechanically stable and thus permit a more reliable movement of the detector.

Moreover, the detector can be movable on the positioning device by way of a motor. Preferably, the detector in this case can be moved in an automated manner by way of a computation unit controlling the motor. This, apart from a more reliable movement to the recording positions, also permits an automatic movement of the detector, so that several projection pictures can be created without the intervention of a user.

An evaluation of the projection pictures taken or recorded by the detector is preferably effected by way of an evaluation unit which can also be part of the computation unit. The evaluation unit for this computes a three-dimensional representation of the object to be imaged and issues this at an output unit, such as on a screen for example, in a manner recognisable to the user. Thus two-dimensional and three-dimensional recordings can thus be created with the detector arrangement. For this, typically several recordings of the object at different angles are combined for creating three-dimensional recordings.

Preferably, the detector is rotated along a spatial axis and/or about a spatial axis. The spatial axis can hereby be a translatory axis, a rotatory axis or a freely shaped axis. The rotatory axis for this can run perpendicularly or parallel to a surface normal of the detector. This permits a particularly simple transformation of detector coordinates and thus a simple and rapid computation of the reconstructions. Inasmuch as a movement is effected about several spatial axes, these in a particularly preferred manner lie orthogonally to one another in a plane.

In a particularly advantageous further development, the detector can be rotatable about a central axis by way of the movement on the positioning device. The surface normal of the detector in this case always has the central axis which lies outside the positioning device, which permits a simple transformation and thus a rapid processing of recorded data. Typically, the central axis runs through a middle point of the object to be imaged.

The detector arrangement can also comprise at least two detectors which are movable independently of one another, in order to achieve as large as possible variability and to simultaneously obtain as many X-ray pictures as possible.

The detector itself is typically a flat detector, i.e. a length and a width of the detector are larger than a depth of the detector. Moreover, the detector is preferably a solid-state detector. This detector type is common for a multitude of applications. A longitudinal axis of the detector and a transverse axis of the detector preferably lie in the imaging surface.

In an advantageous further development, the positioning device is positionable in a spatially fixed manner with regard to the object to be imaged. Thus the position of the detector is determined by two possible movements, specifically the movement of the detector on the positioning device and the movement of the detector together with the positioning device.

A linear displacement drive in combination with a rotation drive for positioning the detector arrangement or the detector can also be used instead of a rail system. A variable positioning of the detector on the positioning device is rendered possible by way of this.

A rest for receiving an object to be imaged by X-ray pictures, in an interior comprises a detector arrangement with the described features. Alternatively, a detector arrangement with the described features can also be attached on an outer side of the rest in a removable manner. A mechanical stability is increased due to a fixed connection of the detector arrangement to the rest. In contrast, a removable fastening has an increased variability.

An X-ray device comprises an X-ray source, an evaluation unit for computing two-dimensional and/or three dimensional reconstructions from the recorded X-ray pictures and a detector arrangement with the previously described features. This permits a recording and further processing of projection pictures from different angles. The X-ray source is preferably fastened on a stand arm and is freely movable into arbitrary positions. Preferably, the X-ray source is fastened on a robot or robot arm, which is fastened on a ceiling of a room. This makes sense for reasons of sterility and spatial requirement.

In a further advantageous further development, the X-ray device also comprises the rest with the previously described characteristics. Thus a complete system is present, with which the object to be imaged can also be securely held.

In a particularly preferred manner, the detector arrangement and/or the X-ray source is arranged in each case on an arm of a robot which is movable by way of three robot arm axes. The arm hereby can be movable in at least three degrees of freedom, in particular in four degrees of freedom, preferably in five degrees of freedom, particularly preferably in six degrees of freedom. The six degrees of freedom hereby include three translatory and three rotatory degrees of freedom. The X-ray source and the X-ray detector can be positioned arbitrarily to one another by way of the movement carried out by the robot or robots, so that projection pictures of the object to be imaged can be obtained amongst a multitude of freely selectable angles.

A method for recording X-ray pictures with an X-ray device, which comprises the X-ray source with the previously described features and the detector arrangement with the previously described features, has several steps. In one step, the X-ray source is moved into several positions for recording individual X-ray pictures of the object to be imaged. The detector arrangement and/or the detector are moved for recording the individual X-ray pictures. Finally, a plurality of projection pictures recorded by the detector is recorded as X-ray pictures by way of the evaluation unit. This serves for the reconstruction of a three-dimensional model of the object to be imaged.

This permits obtaining recordings of the object to be imaged, from a multitude of perspectives and to further process these into a three-dimensional reconstruction. The mentioned elements can be moved into arbitrary positions to one another by way of the movement of the X-ray source and of the detector arrangement or of the detector to one another.

Preferably, a projection picture is recorded in each in each of the recording positions. This permits an unambiguous assignment of the projection pictures to the recording positions. The detector arrangement and/or the detector, for recording, are preferably moved into a position lying opposite the X-ray source, wherein lying opposite does not necessarily imply an orthogonal incidence of the X-ray beams on the detector. The X-ray source can hit the detector at an arbitrary angle, which significantly simplifies the recording of the projection pictures.

In order to save time, for example during an operation, the X-ray source and the X-ray detectors or detector can be moved simultaneously.

In a preferred manner, several regions of the object and which are to be imaged (which are also indicated as a target region) are reconstructed by way of projection pictures which to some extent are equal, in order to reduce the beam exposure of the object due to excessively many recordings.

Moreover, one can envisage the X-ray source travelling through a path, preferably a circular-arc-shaped path with an elliptical defection of at least 90°, above the object to be imaged, in order to be able to obtain a multitude of projection pictures on a predefined path, for taking the projections pictures.

Typically, a plurality of projection pictures is put together into a coherent projection or an individual projection picture of the target region to be imaged or of the respective target regions to be imaged, for imaging and reconstructing a larger target region which is to be imaged and which cannot be covered by a single projection picture, and/or a plurality of individual target regions which are possibly distanced to one another. Inasmuch as individual ones of the projection pictures image several of the target regions, these projection pictures can be used several times, in order to obtain a preferably three-dimensional reconstruction of the respectively desired target region. The target region to be imaged can thus be imaged with a reduced number of projection pictures and with a reduced radiation exposure.

The present invention is not limited to the embodiments which have been described up to now. The following embodiments in each case taken on their own also represent inventions. These embodiments can also comprise the already disclosed features of the previously described further developments. In particular, an X-ray device can comprise a detector arrangement according to one of the following embodiments and a method carried out with this X-ray device. The method of course can also be carried out with the detector arrangement according to one of the following embodiments.

A rest for an object to be imaged, preferably in an interior comprises a detector arrangement with a positioning device and with a detector for X-ray radiation, wherein the detector is movable on the positioning device in the rest in at least two spatial axes. The detector can be moved to different points in a simple manner by way of this, without the object to be imaged itself having to be moved.

The two spatial axes preferably lie in a horizontal plane, which simplifies the movement. The positioning device can comprise a rail system, which can also be cascaded. A mechanically stable and reliable guiding of the detector is achieved by way of this. The rail system hereby can have the already described characteristics.

Moreover, the detector can also be moved by a linear drive, in particular a motor, with the previously described characteristics. This permits an automated displacement of the detector and a defined movement to selected positions.

The detector arrangement can be arranged below the rest as a separate component. In a preferred manner, the positioning device in this case is arranged in a housing, whilst the detector can be displaced in at least two spatial axes which preferably lie in a horizontal plane. The housing can comprise the already described features, thus preferably consist of a material which is transparent to X-ray radiation, or comprise such a material. The detector arrangement is thus removably attachable onto different rests and can be moved or exchanged in a simple manner.

Preferably, the detector is arranged in a housing such as a support foot, which is movable below the rest and which can be moved into at least one, preferably two spatial directions. The housing for this can be connected to the rest via a linear bearing, and particularly preferably have no contact to a floor, on which the rests stands.

The positioning device can comprise at least one side element, into which the detector can preferably be moved. This side element is typically arranged in a manner such that it can be removably attached to a middle part or folded away from this. The side element can also lie in a plane with a middle part of the positioning device. An imaging surface runs along the middle part and in the case of the side element attached onto the middle part can also continue along the side element as a side imaging surface.

The detector is preferably a flat detector. In a particularly preferred manner, the detector is a solid state detector. These are common detector forms which can be constructed in a space-saving manner.

Embodiment examples of the invention are represented in the drawings and are hereinafter explained by way of the figures.

There are shown in:

FIG. 1 a lateral view of an X-ray device, which comprises a X-ray source, a detector arrangement and a rest,

FIG. 2 a lateral view of a further embodiment example of the X-ray device, which is rotated by 90° to the view represented in FIG. 1,

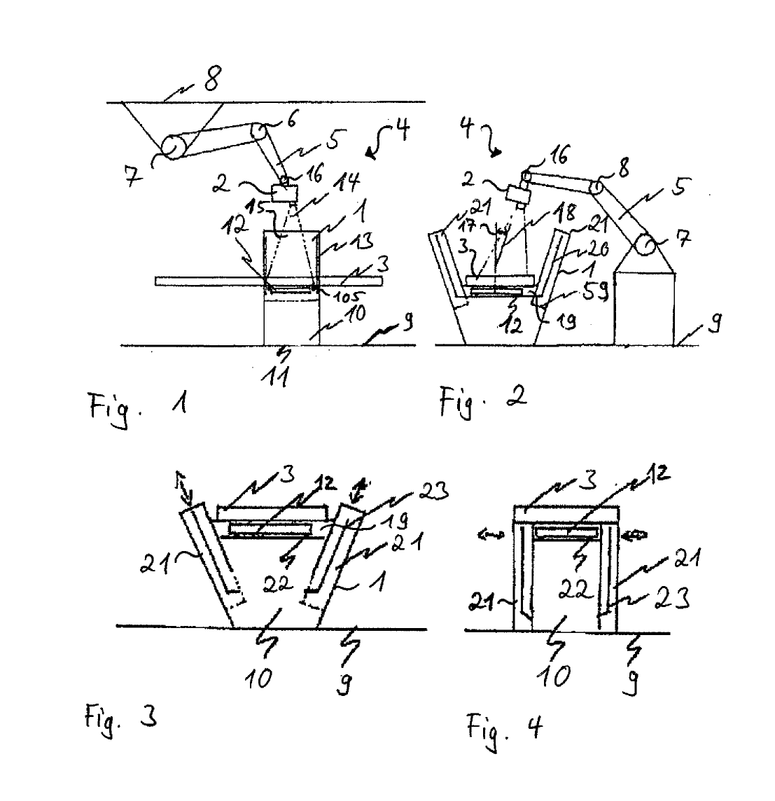

FIG. 3 a view of the detector arrangement which corresponds to FIG. 2, with side elements which can be folded away,

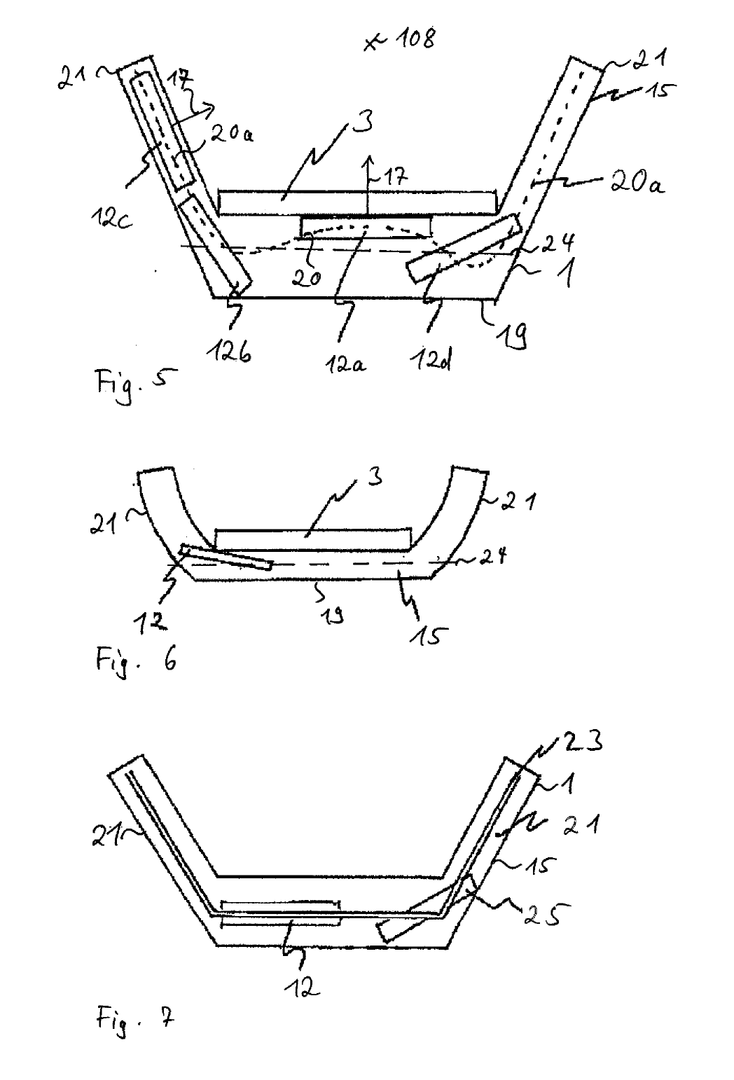

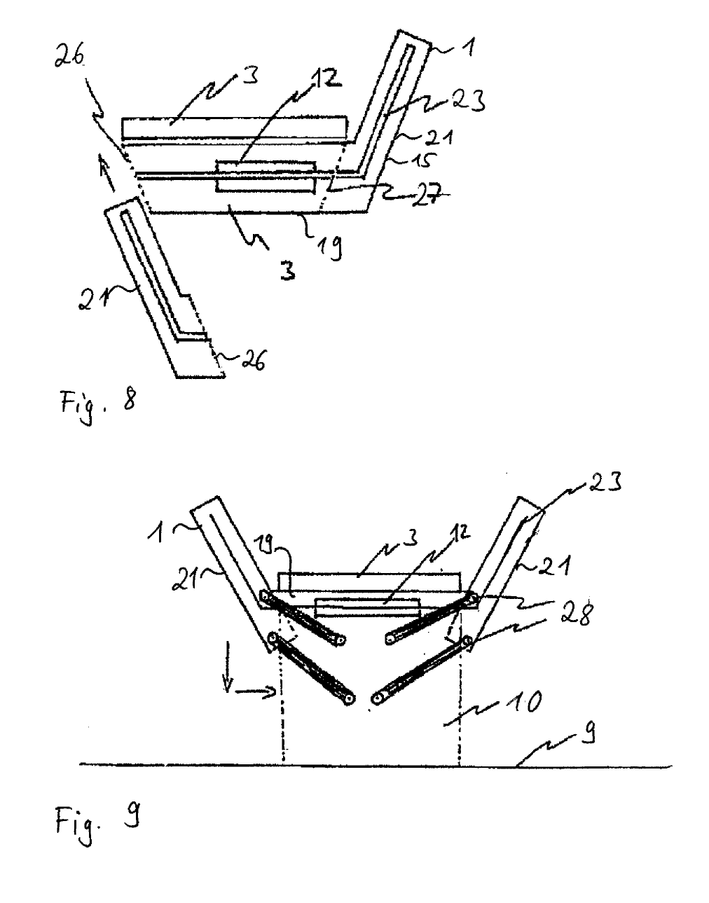

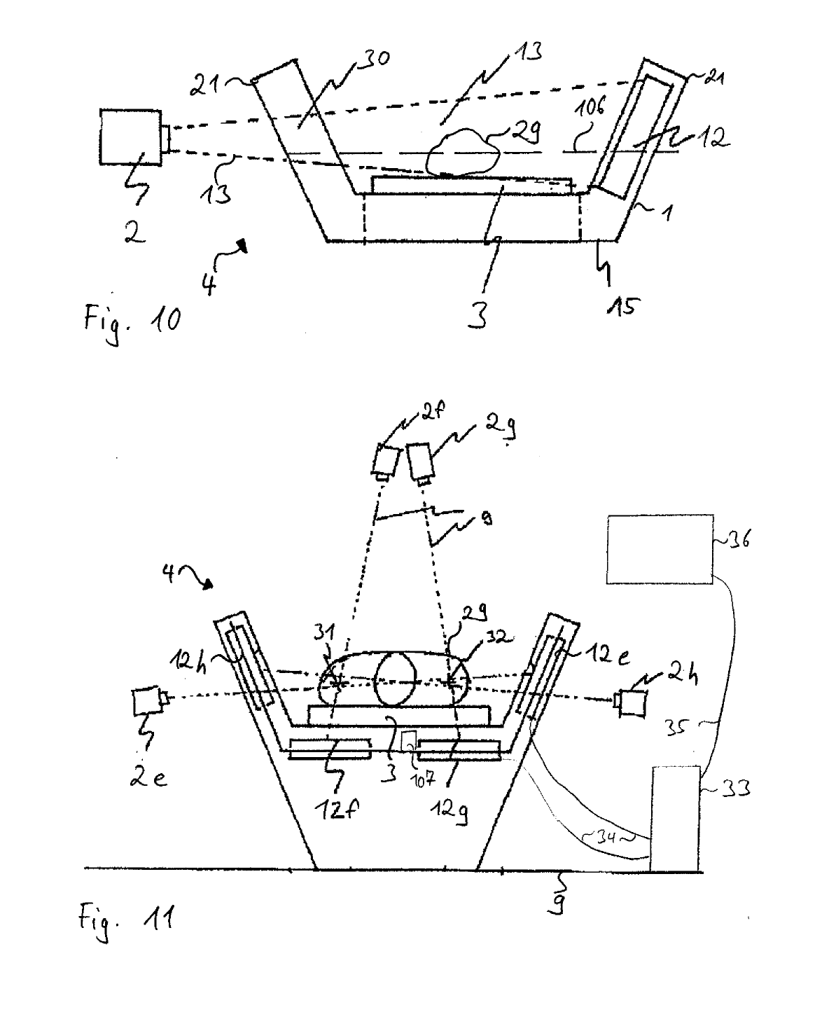

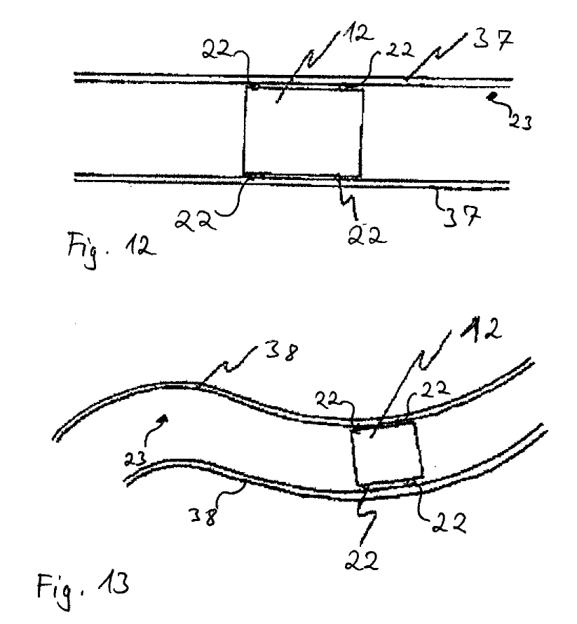

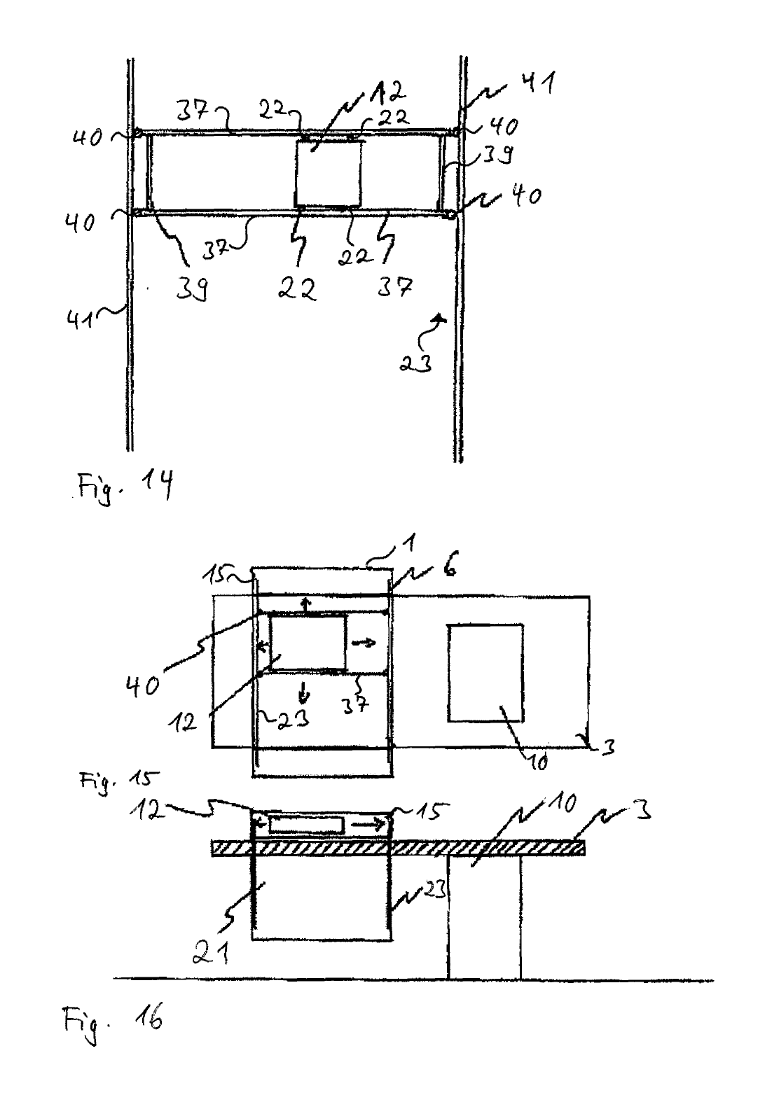

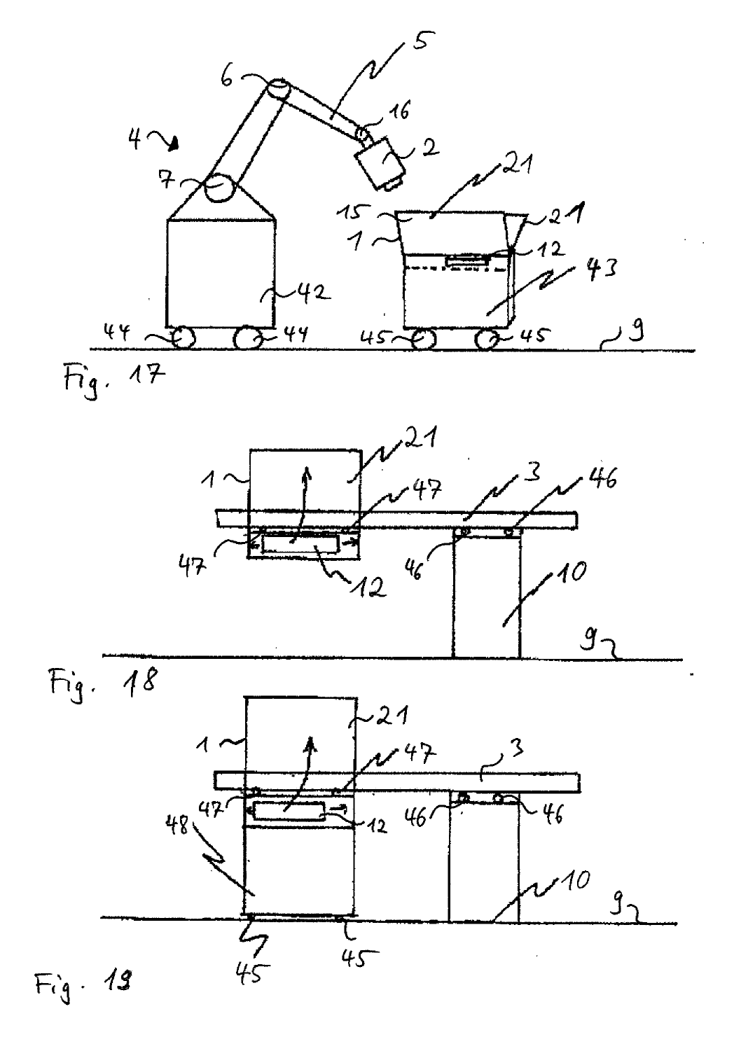

FIG. 4 the detector arrangement shown in FIG. 3, with folded-away side elements,

FIG. 5 a view of a further embodiment of the detector arrangement which corresponds to FIG. 3, with a detector moved into different recording positions,

FIG. 6 a further embodiment of the detector arrangement which is shown in FIG. 5, with arcuate side elements,

FIG. 7 a view of a further embodiment example of the detector arrangement, which corresponds to FIG. 3, with a rail system and two detectors,

FIG. 8 a view of a further embodiment example of the detector arrangement which corresponds to FIG. 3, with a removed side element.

FIG. 9 a lateral view of an embodiment of the detector arrangement which corresponds to FIG. 3, with a guiding of the side elements,

FIG. 10 a view of an embodiment of the X-ray device which corresponds to FIG. 5, with which device the detector and the X-ray source are movable into several recording positions,

FIG. 11 a view of an embodiment example of the X-ray device which corresponds to FIG. 10, with several positions of the detector and of the X-ray source,

FIG. 12 a plan view of a straight-lined rail system of the detector arrangement,

FIG. 13 a view of a rail system which corresponds to FIG. 12, with arcuate rails,

FIG. 14 a view of a cascaded rail system which corresponds to FIG. 12,

FIG. 15 a plan view of a detector arrangement fastened on the rest, wherein the detector is movable above the rest,

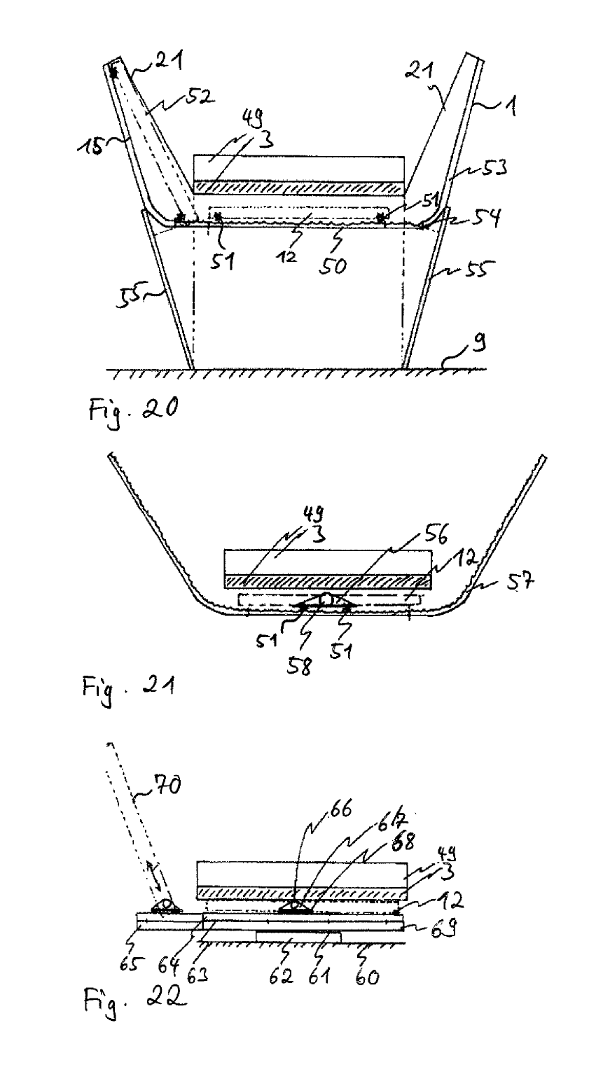

FIG. 16 a lateral view of the detector arrangement shown in FIG. 15,

FIG. 17 a lateral view of a further embodiment of the X-ray device with a movable X-ray source and a movable detector arrangement,

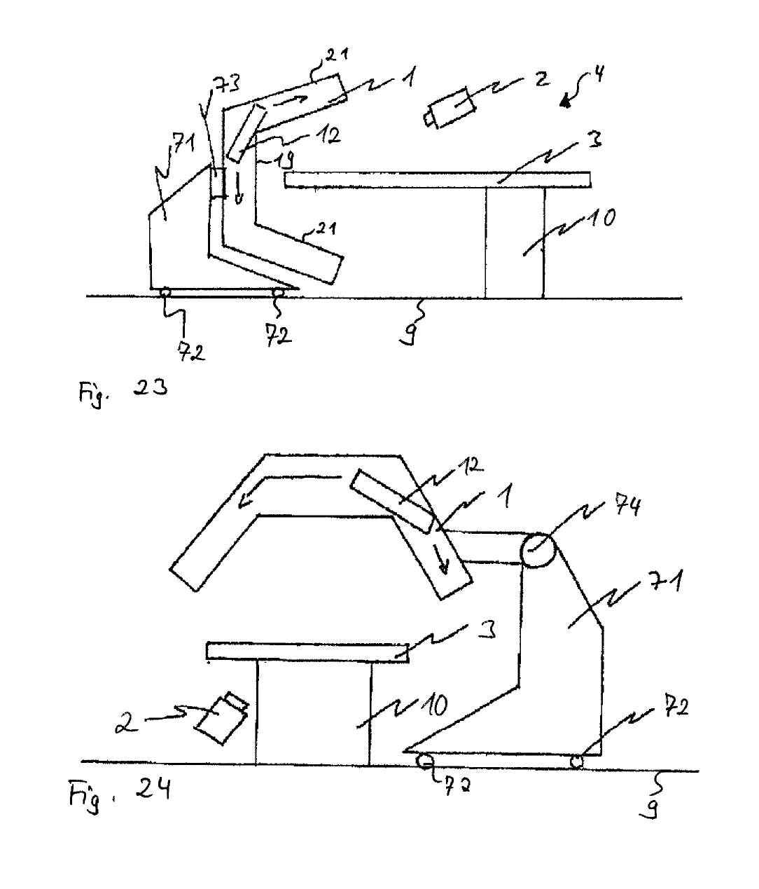

FIG. 18 a lateral view of a further embodiment of the detector arrangement fastened on the rest, with which arrangement the detector is movable below the rest

FIG. 19 a representation according to FIG. 18, of a further embodiment of the detector arrangement displaceably fastened on the rest,

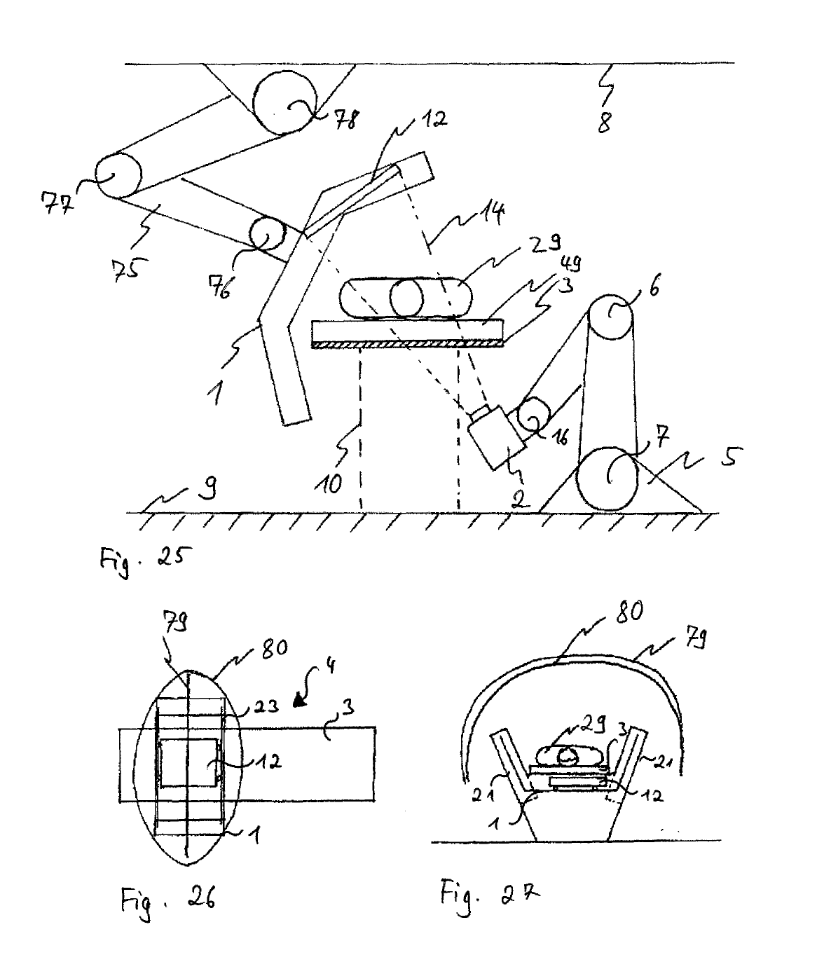

FIG. 20 a view according to FIG. 3, of an embodiment of a the detector arrangement with a guidance by way of a pinion,

FIG. 21 a view corresponding to FIG. 20, of an embodiment of the detector arrangement with a carriage,

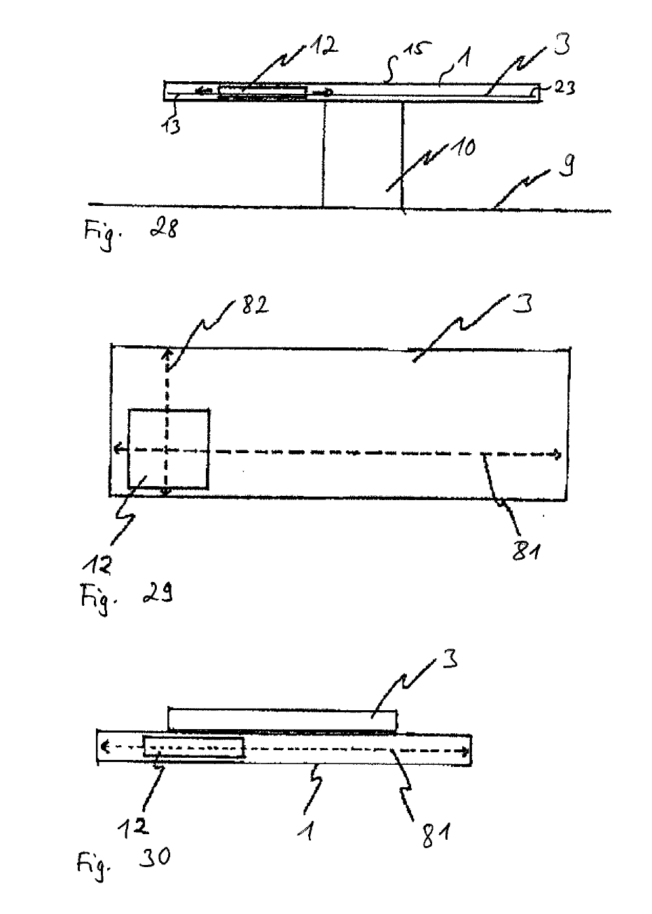

FIG. 22 a representation according to FIG. 21, of an embodiment of the detector arrangement of FIG. 21 with a linear drive,

FIG. 23 a lateral view of an embodiment of the X-ray device, with which the detector arrangement is rotatably attached on a movable vehicle,

FIG. 24 a view corresponding to FIG. 23, of an embodiment of the X-ray device, with which the detector arrangement is arranged above the rest,

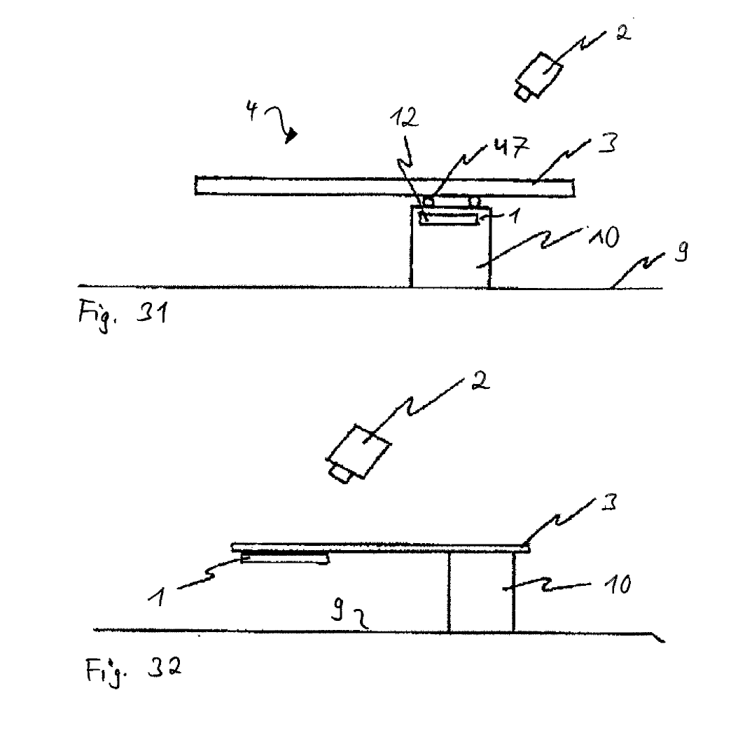

FIG. 25 a lateral view of a further embodiment of the X-ray device, with which the X-ray source and the detector arrangement are in each case fastened on robot arms and are freely positionable,

FIG. 26 a plane view of a recording path of the X-ray source for travelling to several recording positions,

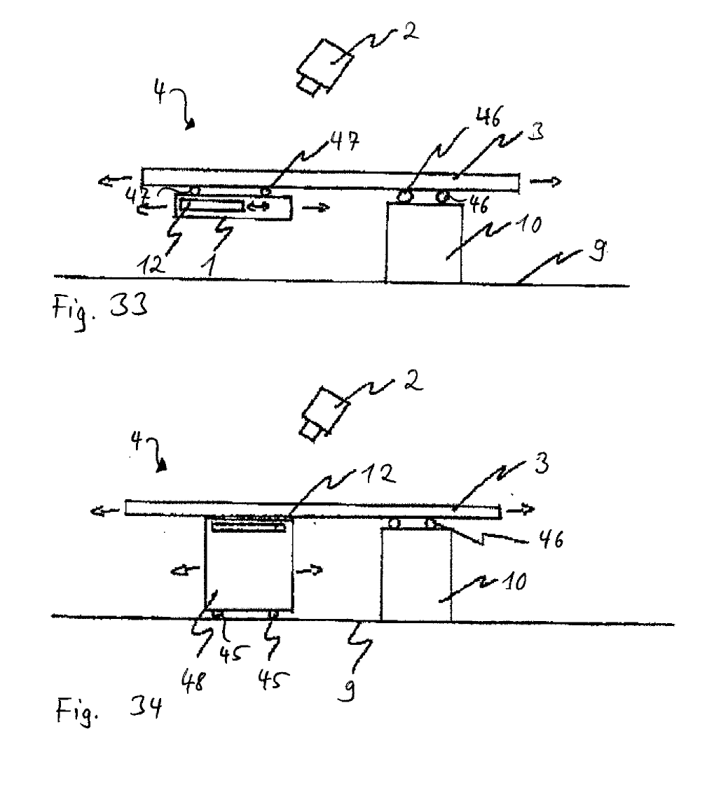

FIG. 27 a lateral view of the recording path shown in FIG. 26,

FIG. 28 a lateral view of the rest with an integrated detector arrangement,

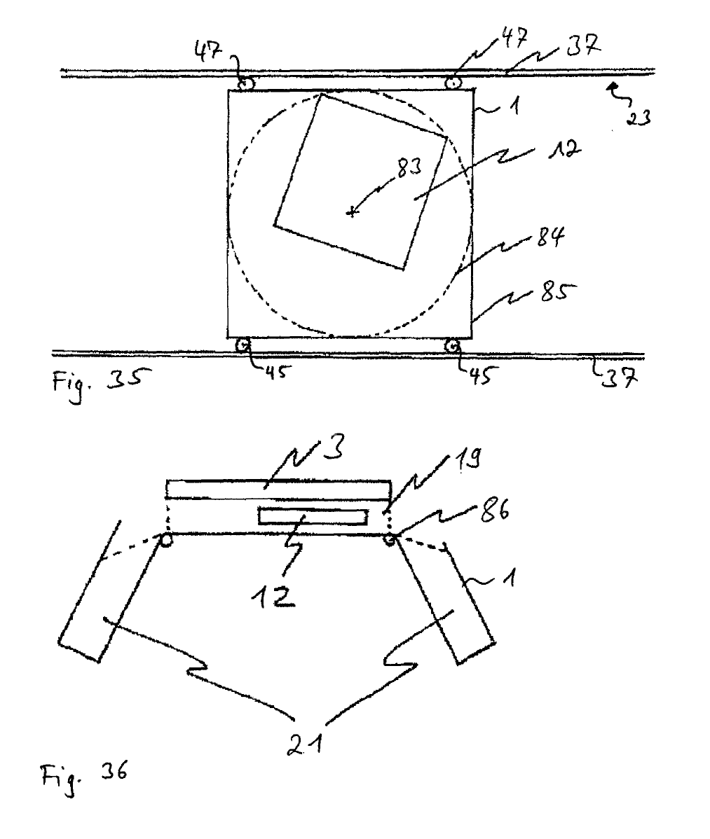

FIG. 29 a plan view of the rest shown in FIG. 28,

FIG. 30 a lateral view of the rest, with a detector arrangement located below the rest,

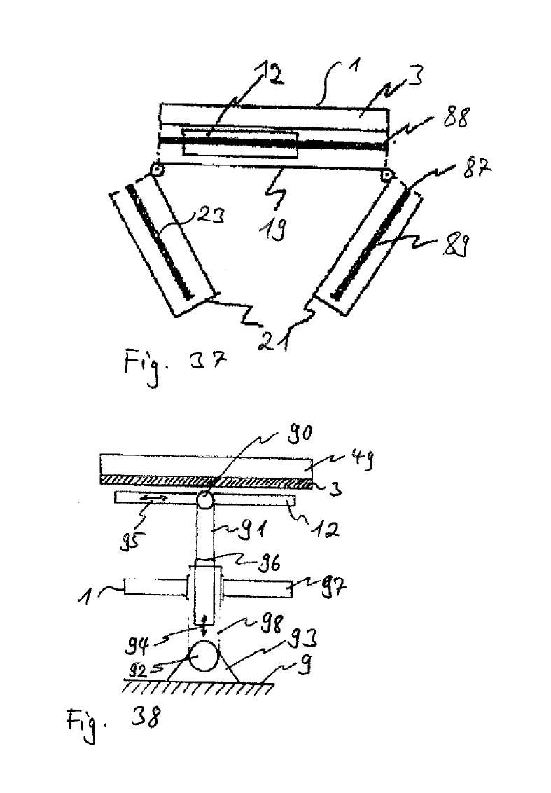

FIG. 31 a lateral view of the rest, with which the detector arrangement is arranged in a table foot located below the rest,

FIG. 32 a lateral view of an embodiment of the rest, with which the detector arrangement is arranged directly below the rest,

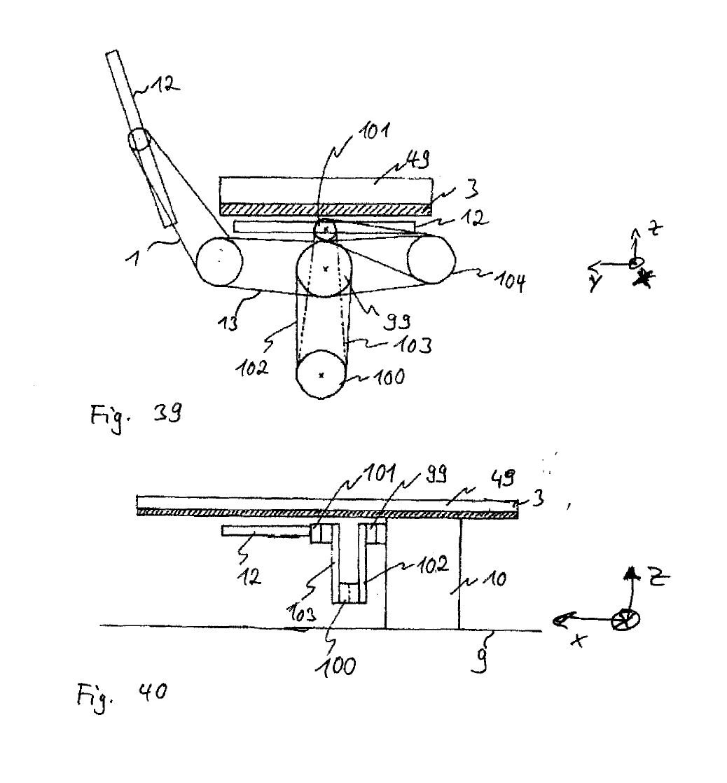

FIG. 33 a lateral view of a further embodiment of the X-ray device, with which the detector arrangement is connected to the rest via a bearing,

FIG. 34 a view according to FIG. 33, of a further embodiment of the X-ray device, with which the detector arrangement is arranged in a movable housing below the rest,

FIG. 35 a plan view of the detector which is rotatably mounted on the rail system,

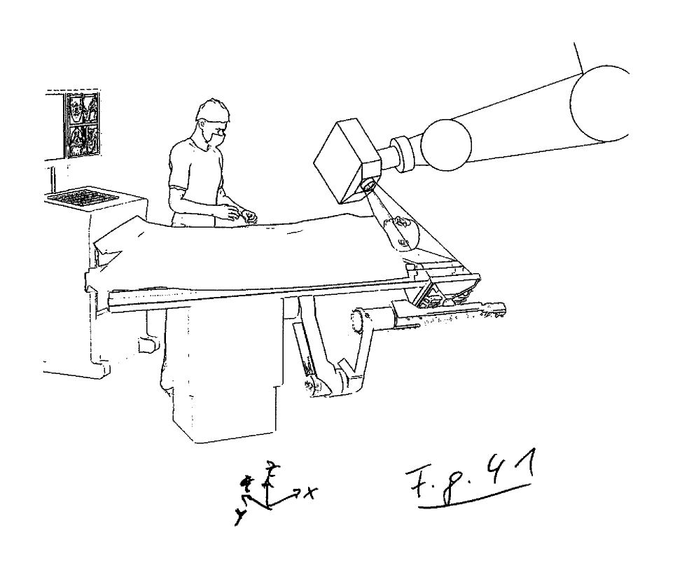

FIG. 36 a lateral view of an embodiment of the detector arrangement with side elements which can be folded onto a middle part,

FIG. 37 a representation according to FIG. 36, with which the positioning device comprises a rail system,

FIG. 38 a lateral view of an embodiment of the detector arrangement, with which the detector can be positioned by a linear displacement drive and a rotation drive,

FIG. 39 a lateral view of an embodiment of the detector arrangement, with which the detector is positionable via an articulated arm,

FIG. 40 a view of the embodiment represented in FIG. 39, which is rotated by 90° as well as

FIGS. 41-45 further views of the embodiment according to FIGS. 39 and 40.

FIG. 1 shows a detector arrangement 1, which together with an X-ray source 2 and a rest 3 forms an X-ray device 4. The X-ray source 2 is fastened on an end of an automatically movable robot arm 5 which is movable via a first joint 6 and a second joint 7 in four spatial directions. In other embodiments, the robot arm 5 can also be moved in six spatial directions, wherein the six spatial directions comprise three translatory and three rotatory spatial directions. The robot arm 5 with an end which is opposite to the X-ray source 2 is fastened on a ceiling 8 of an operating room. The X-ray source 2 is likewise movable in several directions via a third joint 16 and is configured to emit X-ray radiation of different wavelengths and intensities. The robot arm 5 is controlled in its movement via a computation unit, but can also be manually positioned as a stand arm.

A support foot 10 is attached on a floor 9 of an operating room via a fastening 11, for example a screw connection. The rest 3 is horizontally mounted on the support foot 10 in a manner parallel to the floor 9, and this rest in the embodiment example represented in FIG. 1 is an operating table, on which a patient can lie as an object to be imaged by the X-ray device 4.

The detector arrangement 1 below the rest can be removably fastened on the rest 3 by way of a clamping holder 105. Instead of by clamping, the detector arrangement 1 can also be screwed or latched onto the rest 3. Alternatively, the detector arrangement 1 can also be fastened on the rest 3 above it or laterally of the rest 3. The detector arrangement 1 comprises a detector 12, in the shown embodiment example a flat detector which is positioned parallel to the rest 3. Moreover, the detector arrangement 1 comprises a positioning device 13, to which the detector 12 is connected and on which the detector 12 can be moved. The movement of the detector 12 on the positioning device 13 in this embodiment example as well as in the following embodiment examples can be effected manually or by way of a motor. The positioning device 13 comprises a rail system in the embodiment example represented in FIG. 1.

An X-ray cone beam 14 which is emitted by the X-ray source 2 onto the detector 12 arranged lying opposite the X-ray source hits the detector 12 which is designed as a solid state detector. Inasmuch as an object to be imaged lies on the rest 3 within the X-ray cone beam 14, X-ray radiation beams through this object. The X-ray cone beam 14 can be varied in its dimensions by way of a movable aperture in front of the X-ray source 2. The X-ray radiation is absorbed to a different extent depending on materials which are distributed differently spatially in the object, so that X-ray radiation with a spatially different intensity is incident on the detector 12. A projection picture is produced on the detector 12 by way of this, and this picture is led further from the detector 12 to an evaluation unit for further processing and from the evaluation unit is finally represented on an output unit.

The detector arrangement 1 is arranged in a housing 15 of plastic. The housing 15 has a closed surface without holes and completely encloses the detector arrangement 1. The housing 15 is permeable to the X-ray radiation emitted by the X-ray source 2, thus does not or only slightly absorbs the X-ray radiation. Generally, the closer the X-ray source 2 is brought to the detector 12, the larger is the recorded projection picture. The further an origin of the X-ray cone beam 14 is distanced to the target region to be imaged, the greater is the recorded target region, with a simultaneously reduction of the X-ray beam cone angle. Larger regions to be imaged can be reconstructed due to a free positioning of the X-ray source 2 and of the detector 2, by way of a superposition of different target regions contained on the different projection recordings, wherein these larger regions to be imaged are to be understood in particular as regions whose dimensions in at least one direction are greater than a cone beam diameter. Hereby, a part of the projection pictures is used for two or more target regions, by which means a relative dose for the object to be imaged is reduced.

In further embodiment examples, the rest 3 and the detector arrangement 1 can also be designed in a single-part manner, i.e. that the rest 3 at least in a part of its interior comprises the detector arrangement 1.

FIG. 2 shows a further embodiment of the X-ray device 4 in a lateral view, which however has been rotated by 90° with respect to the view represented in FIG. 1. Recurring features in this figure as well as in the subsequent figures are provided with identical reference numerals. The X-ray device 4 in turn comprises the X-ray source 2, the detector arrangement 1 and the rest 3. The X-ray source 2 is arranged on the robot arm 5 which now however is fastened on the floor 9 via a clamping connection. As in the embodiment example shown in FIG. 1, the robot arm 5 comprises three joints 7, 8 and 16. The X-ray cone beam 14 which from the X-ray source 2 hits the detector 12, is now tilted by an angle deviation 18 with respect to a surface normal 17 of the detector 12, and an X-ray central beam does not orthogonally hit the detector 12. The angle deviation 18 describes a deviation of the X-ray central beam of the X-ray cone beam 14 from the surface normal 17. The X-ray central beam hereby is a beam located centrally in the X-ray cone beam 14. The rest 3 is likewise manufactured from a material which is transparent to X-ray radiation, since the X-ray cone beam 14 must pass through the rest 3 before hitting the detector 12.

The detector arrangement 1 comprises a middle part 19 as well as two side elements 21 which can be angled or bent away from the middle part 19. The side elements 21 can be folded away and lowered with resects to the middle part. The middle part 19 is parallel to a plane running through the rest 3 and is horizontal. The middle part 19 in FIG. 2 although being attached below the rest 3, can however also lie above it or laterally of it. The side elements 21 lie opposite one another on the middle part 19 and are connected to this, so that the detector 12 can also be moved into the side elements 21. The side elements 21 in the embodiment example represented in FIG. 3 are open at a lower end 59, in order to be able to remove the detector 12 rapidly out of the detector arrangement 1 and to insert it into this again, for maintenance purposes. The side elements can of course also be closed at the lower end 59 in further embodiment examples.

The rest 3 with a detector arrangement 1 fastened thereon is represented in FIG. 3 in a lateral view corresponding to FIG. 2, without a robot arm 5 and X-ray source 2. The rest 3 in turn rests of the support foot 10 connected to the floor 9 and in contrast to the representation of FIG. 2, the side elements 21 now however are folded away by a movement indicated by arrows, by way of then having been moved next to the middle part 19 of the detector arrangement 1. The rail system 23 in the side elements 21 can be recognised due to the enlarged representation compared to FIG. 2, and this system is arranged centrally between two surfaces delimiting the side elements 21. The detector is guided on the rail system 23 via a motion bearing 22. The side elements 21 for example can be folded on for a recording and subsequently be folded downwards away from the rest 3. A free access to the object to be imaged is given by way of this. The side elements 21 can be folded on again for further recordings, after alignment or other manipulation to the object to be imaged.

FIG. 4 shows the detector arrangement 1 which is represented in FIG. 3, with side elements 21 which have been completely folded away. The side elements 21 are completely below the rest 3, so that a free access to an object located on the rest 3 is given. The side elements 21 for folding on are firstly removed from the position shown in FIG. 4 by way of a movement indicated by horizontal arrows, and subsequently led past next to the rest 3. A stowage below the rest 3 hereby can generally be effected by way of one or more linear and/or one or more rotational movements.

FIG. 5 shows a further embodiment of the detector arrangement 1 in a lateral view corresponding to FIG. 4. The detector arrangement 1 in turn is arranged below the rest 3, but the side elements 21 although being angled with respect to the middle part 19, are however fixedly connected to the middle part 19, thus cannot be folded away. The detector 12 is led parallel to the rail system 23, on an imaging surface 20 which extends along the detector arrangement 1. The imaging surface 20 is led further as a side imaging surface 20a which is angled with respect to the imaging surface 20, in the side elements 21. The imaging surface 20 as well as the side imaging surface 20a can have arbitrary shapes for example planes or saddle surfaces. The side elements 21 are angled by 75° with respect to the imaging surface 20 in the middle part 19 of the detector arrangement 1.

The imaging surface 20 which extends through the middle part 19 and the two side elements 21 is arcuate, and the detector 12 can be moved on the imaging surface 20 or the side imaging surface 20a into several recording positions 12a, 12b, 12c, 12d which are spatially fixed with respect to the rest 3 and the object to be imaged. The detector 12 for this can be moved in the middle part 19 on the imaging surface 20 into several recording positions, such as the recording position 12a. All of the recording positions on the middle part 19, in further embodiments can lie parallel to the rest 3, i.e. the detector 12 in these cases is likewise aligned parallel to the rest 3. The detector 12 can be moved into the side elements 21 by way of a rotation of the detector 12, as is represented in the recording positions 12d and 12b. The recording position 12c characterises an abutment point in the side element 21, i.e. the detector 12 can no longer be moved further. In this recording position 12c, the detector 12 is located completely above the rest 3 and above a middle plane 24 which is arranged centrally between two surfaces of the housing 15 of the middle part 19 and in further embodiments corresponds to the imaging surface 20 of the middle part 19. The imaging surface 20 is not intersected by the detector 12, since the detector 12 with all its components lies above the imaging surface 20. Only the side imaging surface 20 which is angled with respect to the imaging surface 20 is affected by the detector 12 in the recording position 12c.

In further embodiments, the detector arrangement 1 can also be U-shaped or half-round. In the half-round shape, the surface normal 17 of the detector 12 in each position has a central axis 108 which preferably lies parallel to a longitudinal axis of the detector arrangement 1. The central axis 108 in FIG. 5 is drawn in as an intersection point of the surface normal 17 in the recording positions 12a and 12c. The central axis 108 preferably runs through a middle point of the object to be imaged, since the distance to this middle point in the respective recording positions is equal large and thus a further processing of the recorded projection pictures is simplified.

A further embodiment of the detector arrangement 1 with side elements 21 which are arcuate with respect to the middle part 19 are represented in FIG. 6. There is more space for the object to be imaged, in an interior of the detector arrangement 1 which is enclosed by the side elements 21 and the middle part 19, due to the curvature of the side elements 21. The side elements 21 hereby have a width which is smaller than a width of the middle part 19. In further embodiments, the widths however can also be equally large or the width of the side elements 21 can even be larger than the width of the middle part 19. A width of the rest 3 is identical to the width of the middle part 19.

A further embodiment of the detector arrangement 1 corresponding to FIG. 5 is shown in FIG. 7. The detector within the housing 15 is led on the rail system 23 as a positioning device 13, said system going through the middle part 19 and the side elements 21. The rail system 23 is linear and has no curvature, however, the sections of the rail system 23 which are located in the side elements 21 are angled with respect to the section running in the middle part 19. A further detector 25 is now located in the detector arrangement 1, additionally to the detector 12. The further detector 25 can hereby be constructionally the same as the detector 12, but can also have different dimensions or be of a different detector type. The detector 12 and the further detector 25 in the example shown in FIG. 7 are led on the same rail system 23, but the two detectors 12, 25 however can be led on different rail systems. Moreover, the detector 12 is either coupled to the further detector 25 or is moved freely to this. A linear drive for movement can also be applied instead of a rail system. A computation unit can move the detectors 12, 25 via motors and specify the position at which the detectors 12, 25 are most advantageously positioned, in an automated manner. The housing 15 can also serve as a rest 3, i.e. the object to be imaged can also be applied directly on the housing 15 without the otherwise necessary rest 3, and be imaged lying on this housing.

FIG. 8 in a lateral view corresponding to FIG. 5 shows an embodiment of the detector arrangement 1, with which the side elements 21 can be removed from the middle part 19. An open connection region 26 which after the connection of the side elements 21 and the middle part 19 forms a closed connection region 27 encompassed by the housing 15 is arranged in each case on the side elements 21 and the middle part 19 for this.

A further embodiment of the detector arrangement 1 in a lateral view corresponding to FIG. 3, and with side elements 21 led in their movement is shown in FIG. 9. The detector arrangement 1 is connected to the floor 9 via the support foot 10 or a housing container. Guide rods 28 which lead the side elements 21 in their movement which is indicated schematically by the arrows during the folding-on or folding-away are located on the support foot 10. In the folded-on position, the rail system 21 is continuous through the middle part 19 and the two side elements 21.

An embodiment of the X-ray device 4, with which the detector 12 and the X-ray source 2 can be moved into several recording positions, is shown in FIG. 10, in a view corresponding to FIG. 5. The X-ray source 2 in this embodiment example is positioned laterally of the detector arrangement 1. The detector 12 is moved into the side element 21 which is further away from the X-ray source 2, so that the X-ray cone beam 14 firstly radiates through the material 30 of the first of the side elements 21, subsequently the object to be imaged which is located on the rest 3 and finally hits the detector 12 which is located in the second of the side elements 21. Large cone angles for an X-ray picture reproduction can be achieved by the arrangement of the X-ray device 4 which is shown in FIG. 10. The detector 12 at least partly projects beyond a plane 106 which runs through a middle point of the object 29 to be imaged and which lies parallel to a surface of the rest 3 and of the middle part 19 of the detector arrangement 1, in the recording position of the detector which is shown in FIG. 10.

In the embodiment of the X-ray device 4 which in FIG. 11 is shown in a view corresponding to FIG. 10, the detector 12 as well as the X-ray source 2 are shown in several positions. The X-ray source 2 and the detector 12 are preferably simultaneously moved into different recording positions for recording projection pictures of the object 29 which is to be imaged and which in the example shown in FIG. 11 is a patient lying on a rest 3. In one recording position 2e of the X-ray source 2 or a corresponding recording position of the detector 12e, the X-ray source 2 and the detector 12 are located in the positions already represented in FIG. 10. In a recording position 2f of the X-ray source 2 or in a recording position of the detector 12f, the X-ray source 2 is located above the object 29 to be imaged, whereas the detector 12 below the rest 3 below a left half of the object 29 to be imaged lies opposite the X-ray source 2. In the recording position 2g, the X-ray source 2 likewise lies above the object 29 to be imaged, whereas the detector 12 has been moved into a recording position 12g which is located below the object 29 to be imaged. The X-ray source 2 and the detector 12 are arranged in a mirrored manner to the recording positions 2e and 12e, in the recording position 2h and 12h respectively.

Projection pictures of the first target region 31 and of the second target region 32 of the object 29 to be imaged and which are taken in the different recording positions can be led together, in order to reconstruct a three-dimensional model of the respective target region 31, 32 by way of a computation unit 33 as an evaluation unit. For this, the computation unit 33 is connected to the detector 12 via a cable 34. Alternatively, instead of via a cable 34, the projection pictures can also be transferred from the detector 12 to the computation unit 33 in a wireless manner, for example via a radio connection. The computation unit 33 evaluates the projection pictures and computes the reconstruction of the examined target region. The reconstruction is transmitted to an output unit 36, in the present embodiment a screen, either via a further cable 35 or in a wireless manner, and is displayed on the output unit 36. The output unit 36 for this can also be located directly on the detector arrangement 1. Individual projection recordings can also be used for several target regions, given target regions which are imaged several times by way of different projection recordings. In the example shown in FIG. 11, the target region 32 in the recording positions 2e, 2g and 2h are beamed through by X-ray radiation which is emitted from the X-ray source 2. Also several projection recordings can be composed into a coherent projection recording in the case of a target region which is larger than the applied detector 12. A motor 107 which is connected via a cable to the computation unit 33 and, activated by this, initiates the movement of the detector 12, is provided in the positioning device 13 below the rest 3 in the middle part 19 of the detector arrangement 1, for moving the detector 12.

FIG. 12 shows a plan view of a straight-lined rail system 23 of the detector arrangement 1 as can be applied in the previously described embodiments. The detector 12 is mounted on the rail system 23 via the motion bearing 22 which is attached on the detector 12 at both sides and which is a rail bearing in the represented embodiment example. In each case, a straight rail 37 of the rail system 23 runs above and below the detector 12. In each case, two of the motion bearings 22 are led on one of the two straight rails 37. The motion bearings 22 as well as the straight rails 37 are of plastic, but in further embodiment examples can also be of the most varied of materials such as metal or carbon-fibre-reinforced plastic such as carbon composite.

An arrangement of rail systems 23 and detector 12 which corresponds to FIG. 12 is likewise shown in a plan view in FIG. 13. In contrast to the embodiment example shown in FIG. 12, the detector 12 however is not led on straight rails 37 via the motion bearing 22, but on arched rails 38 so that the rail systems 23 has an arched course.

The rail system 23, as is shown in FIG. 14 a plan view corresponding to FIG. 12, can also be constructed in a cascaded manner. A middle part of the cascaded rail system 23 with regard to its construction corresponds to the system which is represented in FIG. 12. This arrangement is supported by two struts 39 and is led on further straight rails 41 via in each case two further rail bearings 40 which are arranged at both ends in the neighbourhood of one of the struts 39. Arched or freely shaped rails can of course also be used instead of straight rails 37, 41. In each case two of the further rail bearings 40 are hereby movable or traversable on one of the further straight rails 41. The detector 12 can be moved almost arbitrary in different spatial directions due to the superposition of several rail systems. Spatial axes, about which the one movement is effected by way of the represented cascaded rail system 23, here lie in a plane orthogonally to one another.

FIG. 15 in a plan view shows an embodiment of the detector arrangement 1, with which the detector 12 is movable above the rest 3. The rest 3 is connected to the floor 9 via the support foot 12 or a table foot. The detector arrangement 1 is attached on the rest 3. The detector 12 is led on the rail system 23 represented in FIG. 14, within the housing 15 of the detector arrangement 1, and can be moved in the directions indicated by the arrows. In alternative embodiments, the shown detector arrangement 1 can also be attached below the rest 3 or form a part of the rest 3. The detector arrangement 1 comprises side elements 21 which can be folded away and which can be set up from a position located below the rest 3, and lowered again after the recording. The folding-on and folding-away of the side elements 21 in this, as well as in the already presented embodiments can either be effected manually or in an automated manner, wherein the automated method can be controlled by the computation unit 33 as a control unit.

The embodiment example shown in FIG. 15 is represented in a lateral view in FIG. 16. The detector 12 is arranged above the rest 3, but however can also be moved into the side elements 21 folded away laterally of the rest 3. For this, a part of the rail system 23 is also located in the side elements 21.

FIG. 17 in a lateral view shows an embodiment of the X-ray device 4, with which the detector arrangement 1 as well as the X-ray source 2 are arranged in a mobile manner. The X-ray source 2 is attached on the robot arm 5 and is movable via the first joint 6, the second joint 7 and the third joint 16. The robot arm 5 is fastened on a vehicle 42 which is movable via wheels 44 or on a rail system arranged on the floor 9. The detector arrangement 1 has set-up side elements 21 and is assembled on a detector housing container 43 which is likewise movable on the wheels 45 or on a rail system arranged on the floor 9. The housing 15 of the detector arrangement 1 at the same time also serves as the rest 3, on which the object 29 to be imaged is placed. A spatial assignment and/or the localisation of the X-ray source 2 and of the detector 12 to one another can be effected by markers which are attached on one of the two components, wherein the respective other component, thus that without the attached marker, comprises an evaluation unit for evaluating the spatial position.

A further embodiment of the detector arrangement 1 fastened on the rest 3 is shown in FIG. 18 in a lateral view, wherein the detector 12 is displaceable below the rest 3. The rest 3 can be displaced over the support foot 10 via two motion bearings 46 which are arranged between the rest 3 and the support foot 10. The detector arrangement 1 with folded-on side elements 21 is displaceably fastened on the rest 3 via two motion bearings 47. The detector 12 can thus carry out a movement within the positioning device 13 of the detector arrangement 1 and a second movement with the detector arrangement 1 on the rest 3. Hereby, not only can the detector 12 move in a translatory manner, but can also be rotated. In further embodiment examples, the support foot 10 can also be movably arranged on the floor 9. This in particular can be effected if the detector arrangement 1 is connected to the base floor, a wall or the ceiling 8 in a mobile or stationary manner.

FIG. 19 shows a representation of a further embodiment of the detector arrangement 1 which is displaceably fastened on the rest 3, wherein this representation corresponds to FIG. 18. In contrast to FIG. 18, the detector arrangement 1 now is not only displaceably fastened on the rest 3, but is also displaceable on the floor 9 via the detector container 48 and the wheels 45. Otherwise, the embodiment example shown in FIG. 19 corresponds to the example already described in FIG. 18. The side elements 21 in turn are folded on/up and are located above the rest 3.

A representation of the detector arrangement 1 with a guide by way of a pinion 51 is represented in FIG. 20, in a manner corresponding to FIG. 3. The detector arrangement 1 again is arranged below the rest 3. The rest 3 is now provided with a protective layer 49 such as a cushion, which when contaminated can be easily pulled off and cleaned. The protective layer 49 is of a material which is transparent to X-rays. The detector 12 is led on a rack 50 as a positioning device 13, via two equally running pinions 51. The two pinions 51 are each attached at one end of the detector 12. The detector 12 is displaceable on the rack 50 into the side elements 21, via a join location 54 between the rack 50 and a cog bearing guide 53 in the side elements 21. The detector 12 is represented in a recording position 52 in a side element 21, wherein one of the two pinions 51 which is away from the respective side element 21, still rests on the rack 50 and for moving engages into this, whereas the other of the two pinions 51 lies on the cog bearing guide 53 on a lower part of the housing 15. In each case a side wall 55 which terminates in a flush manner with the side element and delimits a space lying below the detector arrangement 1 and above the floor 9 is arranged below each of the side elements 21.

FIG. 21, in a view corresponding to FIG. 20, shows one embodiment of the detector arrangement 1 which corresponds to that shown in FIG. 20, with which however a continuously bent rack 57 is used, on which the detector 12 is led by a carriage 54. The bent rack 57 is straight in a middle part 19 arranged parallel to the rest 3, as well as in the lateral parts angled with respect to this middle part 19, but is bent in the transition region between the parts. The carriage 56 on its lower side comprises two equally running pinions 51 which are designed as cogs. The carriage 56 is connected to the detector 12 via a driven rotation axis 58 and moves the detector 12 due to the toothed engagement of the pinions 51, into the bent rack 57. This linear movement can be combined with a rotation or inclination of the detector 12 about the rotation axis 58.

The detector arrangement 1 represented in FIG. 21 is likewise represented in FIG. 22 in a linear lateral view, but now differs by way of a linear guide 68 which renders the rail system 23 or the rack 50 superfluous.

FIG. 22 shows a representation of the detector arrangement 1 of FIG. 21 and in a manner corresponding to FIG. 21, with a linear drive 64. A combination of linear axes and inclination axes moves the detector 12 below and laterally of the rest 3 into the desired position, so that a rail system 23 can be done away with. For this, a movable assembly surface 60 is arranged parallel to the rest 3, below this rest 3. A deflector carriage 62 which via a guide 61 which in the embodiment example represented in FIG. 22 comprises a ball bearing guide, is connected to a deflector base 69, is assembled onto the movable assembly surface 60 and is located on the movable assembly surface 60. The deflector base 69 is attached between the rest 3 and the deflector carriage 62 and is movable. The deflector and the linear drive 64 are in contact with one another via a connection 63. A linear drive carriage 67 is arranged between the deflector base 69 and the rest 3 and is connected via a linear guide 68 which is connected to the deflector base 69 and the detector 12. The linear drive carriage 67 is movable via a rotation axis 66. The deflector base 69 can be moved by way of a displacement parallel to the rest 3, into a lateral position 65, in which the deflector base 69 at least partly is no longer covered by the rest 3. In this position, the detector 12 can be rotated about the rotation axis 66 of the linear drive carriage 67 by way of this linear drive carriage 67 and thus be moved into a position 70, in which the detector 12 is arranged laterally next to the rest 3 in a manner angled to the rest 3.

An embodiment of the X-ray device 4 is shown in a lateral view in FIG. 23, with which the detector arrangement 1 is rotatably mounted on a movable vehicle 71. The rest 3 is connected to the floor 9 via the support foot 10, but the detector arrangement 1 is no longer fastened on the rest 3. The detector arrangement 1 in the embodiment example represented in FIG. 23, with regard to its features corresponds to the detector arrangement 1 described in FIG. 5 and is fastened on the vehicle 71 via a rotation axis 73. The vehicle 71 is led on the ground 9 via vehicle wheels 72 or alternatively via a rail system, so that the detector arrangement 1 can be positioned freely but is a spatially fixed manner with respect to the rest 3, by way of moving the vehicle 71. The detector arrangement 1 can be rotated via the rotation axis 73, so that for example the side elements 21 lie with the middle part 19 in a plane with the rest 3. Thus projection recordings can be made from several angles by way of the X-ray source 2 which is likewise freely movable with respect to the rest 3 and which the example shown in FIG. 23 by way of example is arranged above the rest 3, and the projection recordings can be evaluated by way of the evaluation unit which is not represented in FIG. 23 and represented at the output unit.

A further embodiment of the X-ray device 4 of FIG. 23 is represented in FIG. 24 in a lateral view corresponding to FIG. 23. The X-ray source 2 is now arranged below the rest 3, whereas the detector arrangement 1 and thus the detector 12 lie above the rest 3. The vehicle 71 instead of the rotation axis 73 has a folding/rotation axis 74 which connects the vehicle 71 to the detector arrangement 1. The detector arrangement 1 can be positioned above the rest 3 by way of folding, by way of the fold/rotation axis 74. The detector arrangement 1 and thus the positioning device 13 as well as the detector 12 can be moved above, below and laterally of the rest 3.

FIG. 25 in a lateral view represents a further embodiment of the X-ray device 4, with which the X-ray source 2 and the detector arrangement 1 are fastened in each case on robot arms 5, 75 and are freely positionable by the robot arms 5, 75 by way of the computation unit 33. The rest 3 in turn is connected via the support foot 10 to the floor 9, on which the robot arm 5 carrying the X-ray source 2 is also fastened. This robot arm 5 can freely position the X-ray source 2 by way of the first joint 6, the second joint 7 and the third joint 16. The detector arrangement 1 and thus also the detector 12 are connected to the ceiling 8 via the robot arm 75. The robot arm 75 however of course can alternatively also be fastened on the floor 9. The robot arm 75 moves the detector arrangement 1 via a first joint 76, a second joint 77 and a third joint 78, so that the detector arrangement 1 can be set in six degrees of freedom, three rotational and three translatory ones. Additionally, the detector 12 can be moved within the detector arrangement 1, so that the X-ray cone beam 14 of the X-ray source 2 penetrates the object 29 to be imaged and hits the detector 12.

FIG. 26 shows a plan view of the X-ray device 4 represented in FIG. 1, with movement paths of the X-ray source 2. The detector arrangement 1 is fastened on the rest 3, wherein the detector 12 is displaceable on the rail system 23 as already described. A circular recording path 79 of predefined positions of the X-ray source 2 is moved through, for recording the projection pictures. In the example shown in FIG. 26, the circular recording path 79 is semicircular above the rest 3, but however encompasses an angle region of at least 90° above the rest 3. Alternatively, the X-ray source 2 can move through an elliptical recording path 80, with which a lateral deflection of the X-ray source 2 in the direction of a longitudinal axis of the rest 3 is carried out additionally to the circular recording path 79. The detector arrangement 1 is not moved, but remains spatially fixed in the set position whilst the X-ray source 2 travels one of the recording paths. Instead, the detector 12 can be moved on the positioning device 13, within the detector arrangement 1. The computation unit 33 for this can control and regulate the movement of the X-ray source 2 and of the detector 12.

FIG. 27 represents the recording paths 79, 80 shown in FIG. 26, in a lateral view. Preferably, a projection recording is taken in each of the recording positions, wherein a superposition of several recordings can take pace for illustrating and representing larger regions which even with an increased distance between the X-ray source 2 and the object 29 to be imaged cannot be reproduced by a single picture.

FIG. 28 shows a lateral view of the rest 3, into which the detector arrangement 1 and thus the positioning device 13 is integrated. The detector 12 is therefore movable on the rail system 23, within the rest 3, which also serves as the housing 15. The rest 3 is connected to the floor 9 via the support foot 10. The arrangement represented in FIG. 28 is represented in a plan view in FIG. 29. The detector 12 is movable within the rest 3 along a longitudinal axis 81 of the rest 3, over the length of the rest 3 and along a transverse axis 82 of the rest 3 over the width of the rest 3. In further embodiments, the detector 12 can also be rotatable within the plane which is defined by the longitudinal axis 81 and the transverse axis 82. The movement of the detector 12 hereby can be effected manually or by way of a motor.

The detector arrangement 1 is arranged below, but parallel to the rest 3, in the embodiment example shown in FIG. 30 in a lateral view. The detector 12 is displaceable within the detector arrangement 1 in two spatial directions, as already shown in the FIGS. 28 and 29.

As is represented in a lateral view in FIG. 31, the detector arrangement 1 and thus the detector 12 can be arranged in the support foot 10 or table foot, in a further embodiment example of the X-ray device 4. The rest 3 is movable on the support foot 10 via motion bearings 47, so that the rest 3 with the object 29 to be imaged and located thereon can be moved relative to the detector arrangement 1. The detector arrangement 1 for this can also comprise the side elements 21 already described above.

A further embodiment example is likewise shown in the lateral view in FIG. 32, with which the detector arrangement 1 is arranged directly below the rest 3 and is movable relative to the rest 3. The rest 3 is fixedly connected to the floor 9 via the support foot 10, and the X-ray source 2 is freely positionable as well as detached from the floor 9.

An embodiment example is shown in FIG. 33 in a view corresponding to FIG. 32, with which the support foot 10 is fixedly connected to the floor 9, but the rest 3 is movable parallel to the support foot 10 via the motion bearings 46. Moreover, the detector arrangement 1 arranged below the rest 3 is connected via the motion bearings 47 to the rest 3 and is displaceable parallel to this. The detector 12 is arranged within the detector arrangement 1 and is movable in two spatial directions parallel to the rest 3.

FIG. 34 in a view corresponding to FIG. 33 shows a further embodiment of the X-ray device 4, with which the detector arrangement 1 is arranged in a movable housing below the rest 3. The rest 3 is connected to the floor 9 via the motion bearings 46 as in FIG. 33 via the support foot 1. The detector arrangement 1 is accommodated in a detector container 48 which is movable on the floor 9 via wheels 45 or a rail system. The detector container 48 for this can be connected to the rest 3 or be moved independently of this. Moreover, the detector container 48 can comprise side elements 21 which can be folded away or removed.

A plan view of the detector 12 which is rotatably mounted on the rail system 23 is shown in FIG. 35. As already represented in FIG. 12, the detector 12 is a flat detector. The detector 12 is led on a plane holder 85 and this holder 85 is connected to one of the straight rails 37 of the rail system 23 via two motion bearings 22 in each case. The detector 12 is rotatable on the holder 85 about a rotation axis 83 which is located centrally in the holder 85. During the rotation, the detector 12 thus travels the circular surface 84 which is an inner circle of the square holder 85. The rotation axis 83 can also be arranged at a different position as well as be arranged in a fixed or variable manner. The rail system 23 can be contained in the detector arrangement 1 which as is shown in FIG. 28, can also be incorporate directly into the rest 3.

FIG. 36 shows a lateral view of an embodiment of the detector arrangement 1 with side elements 21 which can be folded onto the middle part 19. The side elements 21 are arranged on the middle part 19 on both sides and can be folded onto this or away from the middle part 19 via a hinge 86 in each case, so that the side elements 21 bear on the middle part 19 in a flush manner. Alternatively, the side elements 21 can also be pulled out of the positioning device 13, by way of the side elements 12 lying below the middle part 19 and being pulled out when required. The side elements 21 in this case lie parallel to the rest 3, below which the detector arrangement 1 is arranged. In the folded-on condition, the detector 12 can be moved by the middle part 19 into one of the two side elements 21. In a further embodiment, the side elements can also be folded onto the middle part 19 in a manner such that the side elements 21 are angled with respect to the middle part 19.

FIG. 37 shows the detector arrangement 1 which is already represented in FIG. 36, with the rail system 23 of the positioning device. The rail system 23 comprises a rail 88 in the middle part 19, a connection piece 87 for creating a continuous rail on folding up the side elements 21, and a rail 89 which lies in each case in one of the side elements 21. Thus a continuous rail is created by way of folding on the side elements 21 which lie folded away below the middle part 19, on which continuous rail the detector 12 is displaceable into the side elements 21 and the middle part 1 9.

FIG. 38 shows a lateral view of an embodiment of the detector arrangement 1, with which the detector 12 is positionable by way of a linear displacement drive 98 and a rotation drive. The detector 12 is rotatably mounted about a rotation axis 90 on a U-shaped holder 91 of the linear displacement drive 98. Moreover, the detector 12 along the rotation axis 90 can carry out an additional linear movement 95 parallel to the rest 3. The U-holder 91 or mounting of the linear displacement drive 98 is connected via a flange 96 to the actual linear displacement drive 98 and is movable in a vertical linear displacement movement 94. The linear displacement drive 98 can be mounted on a linear axis 97 which runs parallel to the floor 9, for inclining this linear displacement drive 98. The linear displacement drive 98 is connected to the floor 9 via a floor fastening 93. A further rotation axis 92 is provided on the floor fastening 93 and likewise serves for inclining the linear displacement drive 98.

With the embodiment of the detector arrangement 1 which is represented in a lateral view in FIG. 39, the detector 12 is positionable manually or by motor via an articulated arm 104 as a positioning device 13. The positioning device 13 in this case comprises a first arm section 102 and a second arm section 103 which are preferably equally long. The second arm section 103 is connected via a first rotation axis 99 which lies below the rest 3, to a fastening attached below the rest 3, as well as via a second rotation axis 100 to the second arm section 103. The second arm section 103 is moreover connected to the detector 12 via a third rotation axis 101 which runs through a middle point of the detector 12, wherein the detector 12 is rotatable about the third rotation axis 101 and can be positioned at an arbitrary angle to the second arm section 103. In each case rotations of 360° can be carried out about the first rotation axis 99, the second rotation axis 100 and the third rotation axis 101. The detector 12 can be moved laterally and below the rest 3 on account of the three-axis articulated arm design. The articulated arm 104 can additionally be moved via a linear axis, as described in the context of FIG. 38.

The first arm section 102 and the second arm section 103 can be set parallel to one another as well as vertically to the rest 3, in order to hold the detector 12 below the rest 3 and parallel to this. Alternatively, a parking position 104 can be moved to, with which the detector 12 lies parallel to the rest 3, but the first arm section 102 and the second arm section 103 have been moved into a horizontal position. The second arm section 103 is set parallel to the rest 3, and the second arm section 103 is angled by the second rotation axis 100, in order to move the detector 12 laterally to the rest 3.

FIG. 40 in a view which is rotated by 90° with respect to the view of FIG. 39 shows the arrangement represented in FIG. 39. The articulated arm 104 is connected to the support foot 10 which is connected to the floor 9 and on which the rest 3 is arranged. The first arm section 102 and the second arm section 103, just as the detector 12, are arranged below the rest 3 in their base position.

FIGS. 39 and 49 show all features of patent claim 1. FIGS. 41 to 45 are attached only for explanation and serve merely for an even better illustration.

FIG. 41 hereby shows a standing operator on a column (a support foot) with a rest located thereon as well as a patient. An X-ray apparatus (with articulated arm/robot arm) which is located on the ceiling is directed onto the patient, and the X-ray recording is carried out by a detector at the end of the second robot arm (thus on the positioning device 13).

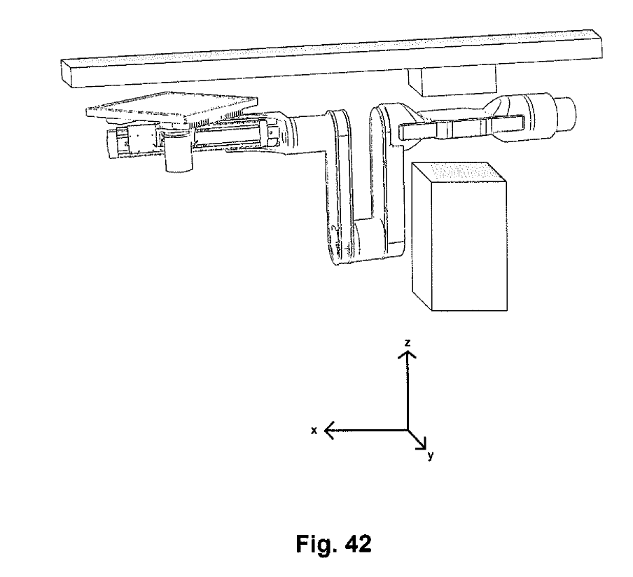

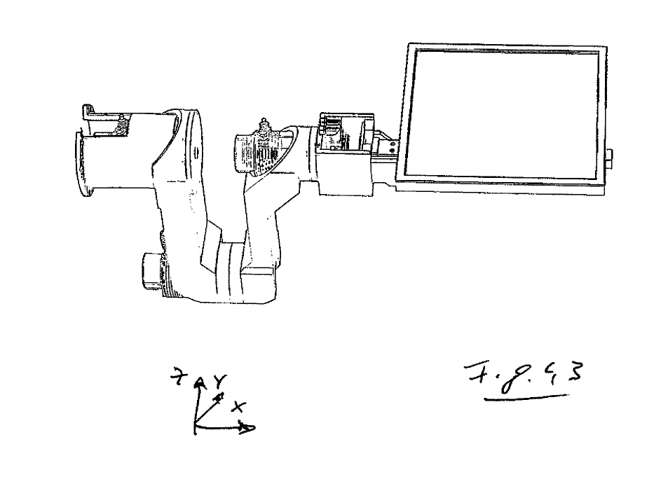

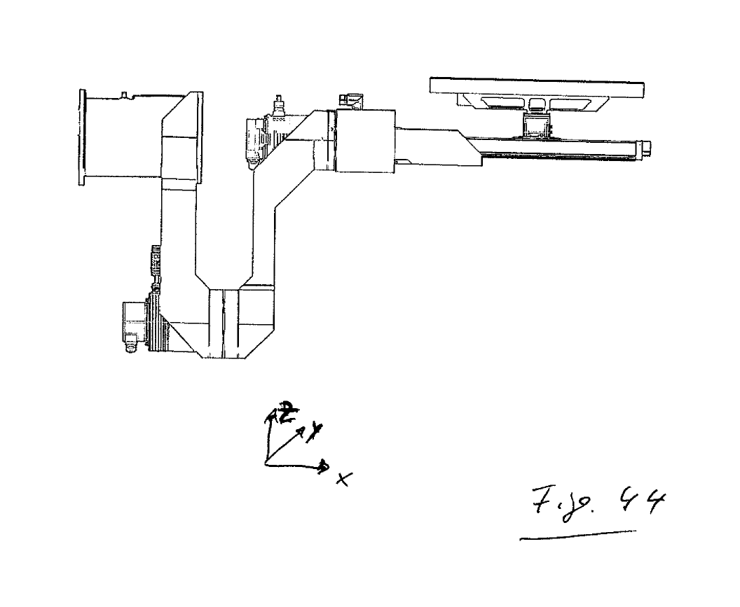

FIG. 42 shows an exploded representation of a support foot 10 of a positioning device 13 as well as a rest 3. Hereby, one can easily see that the positioning device 13 can be arranged essentially between the support foot 10 as well as the rest 3 and thus a retrofitting of the positioning device is conceivable.

FIG. 43 shows a normal position of the positioning device 13 with a detector 12 located at the end of a robot arm/articulated arm.

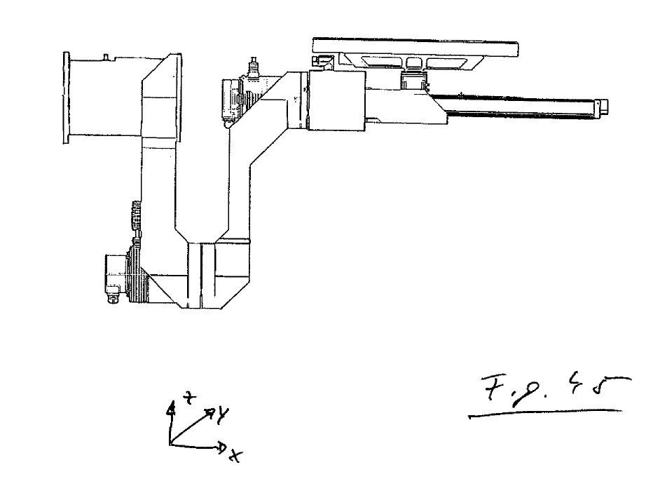

Lateral views of the positioning device are shown in the FIGS. 44 and 45. Hereby, a linear drive at the end of the robot arm is provided centrally with the detector 12 in FIG. 44, whereas in FIG. 45 the detector 12 is displaced to the left (thus in the negative x-direction).

The FIGS. 39 to 45 thus show a detector arrangement for recording X-ray pictures of an object which is to be imaged and which is located on a rest of a table, wherein the table comprises at least one support foot for moving the rest, comprising: a detector for detecting X-ray radiation and a positioning device for moving the detector with respect to the object, wherein the detector can be moved into several recording positions which are spatially fixed with respect to the object, and the positioning device can be fastened on the support foot in a manner such that the positioning device is moved together with the rest, and the positioning device comprises an articulated arm which is movable via at least one rotation axis and/or a linear axis.

Features of the different embodiments which are merely disclosed in the embodiment examples can be combined with one another and individually claimed.

The present intellectual property application amongst other things relates to the following aspects:

1. A detector arrangement for recording X-ray pictures of an object to be imaged, comprising
a detector for detecting X-ray radiation and
a positioning device for moving the detector with respect to the object, wherein the detector is movable on an imaging surface along the positioning device into several recording positions which are spatially fixed with respect to the object,
characterised in that
the positioning device comprises at least one side element which can be angled with respect to the imaging surface
or the positioning device comprise an articulated arm, which is movable via at least one rotation axis and/or a linear axis.
2. A detector arrangement according to aspect 1, characterised in that the detector can be moved into the side element.
3. A detector arrangement according to aspect 2, characterised in that the detector in at least one recording position situated on the side element lies completely outside the imaging surface.
4. A detector arrangement according to one of the preceding aspects, characterised in that the positioning device and the detector are arranged within a housing, wherein the housing preferably comprises a material which is transparent to X-ray radiation and particularly preferably comprises a closed surface.
5. A detector arrangement according to one of the preceding aspects, characterised in that the detector arrangement by way of a holder is removably fastenable on a rest, on which the object to be imaged lies.
6. A detector arrangement according to one of the preceding aspects, characterised in that the at least one side element is removably attachable onto the positioning device, can be folded away from the positioning device and/or can be pulled out of the positioning device.
7. A detector arrangement according to aspect 5 or aspect 6, characterised in that the side element projects beyond a plane which runs parallel to the rest through the object to be imaged.
8. A detector arrangement according to one of the preceding aspects, characterised in that the positioning device comprises at least one rail system, a linear drive, an articulated mechanism or a combination of rail system, a linear drive and an articulated mechanism, wherein the detector is mounted on the rail system.
9. A detector arrangement according to one of the preceding aspects, characterised in that the positioning device is movable below, above and/or laterally to the object.
10. A detector arrangement according to one of the preceding aspects, characterised in that detector is displaceable on the positioning device by way of a linear drive.
11. A detector arrangement according to one of the preceding aspects, characterised in that the detector is movable by a rack and a pinion on a bearing guide of the positioning device.
12. A detector arrangement according to one of the preceding aspects, characterised in that the detector is movable on the positioning device by way of a motor, wherein preferably the detector is movable in an automated manner by a computation unit controlling the motor.
13. A rest for recording an object to be imaged by way of X-ray pictures, characterised in that the rest in an interior comprises a detector arrangement according to one of the preceding aspects, or a detector arrangement according to one of the preceding aspects is removably attachable onto an outer side of the rest.
14. An X-ray device, comprising an X-ray source, an evaluation unit for computing two-dimensional and/or three dimensional reconstructions from the recorded X-ray pictures and a detector arrangement according to one of the aspects 1 to 12.
15. A X-ray device according to aspect 14, characterised in that the detector arrangement and/or the X-ray source is arranged in each case on an arm of a robot which is movable by way of at least three robot arm axes, wherein the arm is movable in at least three degrees of freedom, in particular four degrees of freedom, preferably five degrees of freedom, particularly preferably six degrees of freedom.
16. A method for recording X-ray pictures with an X-ray device, which comprises a X-ray source and a detector arrangement according to one of the aspects 1-12, comprising the steps of:
moving the X-ray source for recording individual X-ray pictures of the object to be imaged, into several recording positions,
moving the detector arrangement and/or the detector for recording the individual X-ray pictures;
recording a plurality of recorded X-ray pictures by way of an evaluation unit for the reconstruction of a three-dimensional model of the object to be imaged.
   17. A method according to aspect 16, characterised in that for recording the prosecution pictures, the X-ray source passes through a path, preferably a circular-arc-shaped path or a circular-arc-shaped path with an elliptical deflection of at least 90° above the object to be imaged.

18. A method according to aspect 16 or aspect 17, characterised in that a plurality of projection pictures is composed into a coherent projection of the target region to be imaged or of the respective target regions to be imaged, for imaging and reconstructing a larger target region or a plurality of target regions.

19. A method according to aspect 18, characterised in that individual ones of the projection pictures are used several times for reconstructing several of the target regions which are imaged by several projection pictures.

### LIST OF REFERENCE NUMERALS

1 detector arrangement
2 X-ray source
2e recording position
2f recording position
2g recording position
2h recording position
3 rest
4 X-ray device
5 robot arm
6 first joint
7 second joint
8 ceiling
9 floor
10 support foot
11 fastening
12 detector
12a recording position
12b recording position
12c recording position
12d recording position
12e recording position
   12f recording position
12g recording position
12h recording position
13 positioning device
14 X-ray cone beam
15 housing
16 third joint
17 surface normal of the detector
18 angle deviation
18 middle part of the detector arrangement
20 imaging surface
20 side imaging surface
21 side element
22 motion bearing
23 rail system
24 middle plane
25 further detector
26 open connection region
27 closed connection region
28 guide rods
29 object to be imaged
30 beamed-through material of the side element
31 first target region
32 second target region
33 computation unit
34 cable
35 further cable
36 output unit
37 straight rail
38 bent rail
39 strut
40 further rail bearing
41 further straight rails
42 vehicle of the X-ray source
43 detector housing container
44 wheels
45 wheels
46 motion bearing support foot/rest
47 motion bearing rest/detector arrangement
48 detector container
49 protective layer
50 rack
51 pinion
52 recording position
53 cog sliding guide
54 join location
55 side wall
56 carriage
57 bent rack
58 driven rotation axis
59 lower side of side part
60 movable assembly surface
61 guide
62 deflector carriage
63 connection
64 linear drive
65 lateral position
66 rotation axis
67 linear drive carriage
68 linear guide
69 deflector base
70 angled position of the detector
71 vehicle
71 vehicle wheels
73 rotation axis
74 fold/rotation axis
75 robot arm
76 first joint
77 second joint
78 third joint
79 circular recording path
80 elliptical recording path
81 longitudinal axis of the rest
82 transverse axis of the rest
83 rotation axis
84 travelled region
85 holder
86 hinge
87 connection piece
88 rail in the middle path
89 rail in the side element
90 rotation axis
91 U-shaped holder
92 further rotation axis
93 floor fastening linear displacement drive
94 movement direction of the linear displacement drive
95 linear movement
96 flange
97 linear axis
98 linear displacement drive
99 first rotation axis
100 second rotation axis
101 third rotation axis
102 first arm section
103 second arm section
104 articulated arm in parked position
105 clamping holder
106 plane running through the middle point of the object
107 motor
108 central axis

1. A detector arrangement for recording X-ray pictures of an object which is located on a rest of a table and which is to be imaged, wherein the table comprises at least one adjustable support foot for moving the rest, the detector arrangement comprising:
a detector for detecting X-ray radiation; and
a positioning device for moving the detector with respect to the object,
wherein the detector is movable into several recording positions which are spatially fixed with respect to the object;
wherein the positioning device is fastenable on the support foot in a manner such that the positioning device is moved together with the rest; and
wherein the positioning device comprises an articulated arm which is movable via at least one rotation axis and/or a linear axis.

2. The detector arrangement according to claim 1, wherein the positioning device is fastenable between the support foot and the rest.

3. The detector arrangement according to claim 1, wherein the rest consists at least in regions of a material transparent to X-rays.

4. The detector arrangement according to claim 1, wherein the support foot is designed such that the rest and the positioning device together are adjustable in height and/or in inclination.

5. The detector arrangement according to claim 4, wherein the inclination is adjustable in one or two spatial directions (x and/or y).

6. The detector arrangement according to claim 1, wherein the rest is held on exactly one support foot.

7. The detector arrangement according to claim 1, wherein the rest and the positioning device are positioned with respect to the support foot in a manner such that an operator can accommodate his legs below the rest.

8. The detector arrangement according to claim 1, wherein the rest has a longitudinal direction (x), and the rest with respect to the support foot is movable in this longitudinal direction (x) either together with the positioning device or independently of the positioning device.

9. The detector arrangement according to claim 1, wherein the positioning device comprises a robot arm with at least three robot arm axes for positioning the detector.

10. The detector arrangement according to claim 9, wherein three of the robot arm axes are arranged parallel to one another.

11. The detector arrangement according to claim 9, wherein the detector by way of a positioning device can be positioned additionally translatorily (preferably in the x-direction and/or y-direction) with respect to the rest.

12. The detector arrangement according to claim 11, wherein the translatory positionability is given in at least one spatial direction (x).

13. The detector arrangement according to claim 12, wherein the spatial direction is a longitudinal direction (x) of the rest and/or is parallel to at least one robot arm axis.

14. The detector arrangement according to claim 12, wherein the translatory positionability is given in two or three spatial directions.

15. The detector arrangement according to claim 1, wherein the detector is designed in an essentially two-dimensional manner and in an initial position can be positioned parallel below the rest such that the legs of an operator can be arranged between the floor and the detector.

16. The detector arrangement according to claim 1, wherein the positioning device is designed in a manner such that the detector is movable from at least two sides of the rest from below the rest to above the rest.

17. The detector arrangement according to claim 1, wherein the positioning device is movable below, above and/or laterally to the object.

18. The detector arrangement according to claim 1, wherein the detector is movable on the positioning device by way of a linear drive.

19. The detector arrangement according to claim 1, wherein the detector is movable on the positioning device by way of a motor, wherein preferably the detector is movable in an automated manner by way of a computation unit controlling the motor.

20. An X-ray device comprising an X-ray source, an evaluation unit for computing two-dimensional and/or three-dimensional reconstructions from the recorded X-ray pictures, and a detector arrangement according to claim 1.

21. The X-ray device according to claim 20, wherein the X-ray source is arranged on a movable arm of a robot, wherein the robot arm can be fastened on a ceiling of a room.

22. The X-ray device according to claim 20, wherein the detector arrangement and/or the X-ray source is arranged in each case on an arm of the robot which is movable by way of at least three robot arm axes, wherein the arm is movable in at least three degrees of freedom.

23. A method for recording X-ray pictures with an X-ray device which comprises an X-ray source and a detector arrangement according to claim 1, comprising the steps:
moving the X-ray source for recording individual X-ray pictures of the object to be imaged, into several recording positions;
moving the detector arrangement and/or the detector for recording the individual X-ray pictures; and
recording a plurality of recorded X-ray pictures by way of an evaluation unit for the reconstruction of a three-dimensional model of the object to be imaged.

24. The method according to claim 23, wherein the X-ray source travels through a path, preferably a circular-arc-shaped path or a circular-arc-shaped path with an elliptical deflection of at least (90°) above the object to be imaged, for recording of the projection pictures.

25. The method according to claim 22, wherein a plurality of the projection pictures are composed into a coherent projection of the target region to be imaged or of the respective target regions to be imaged, for imaging and reconstructing a larger target region or a plurality of target regions.

26. The method according to claim 25, wherein individual ones of the projection pictures are used several times for reconstructing several of the target regions which are imaged by several projection pictures.
